(19) United States
(12) Patent Application Publication
(10) Pub. No.: US 2006/0005293 A1
(43) Pub. Date: Jan. 12, 2006

(54) Safety harnesses

(76) Inventors: John R. Frey, Franklin, PA (US); Preston L. Anderson, Cranberry, PA (US); Tracy H. Lang, Mercer, PA (US)

Correspondence Address: BARTONY & HARE, LAW & FINANCE BUILDING, SUITE 1801, 429 FOURTH AVENUE, PITTSBURGH, PA, 15219, US

(21) Appl. No.: 11/179,228

(22) Filed: Jul. 12, 2005

Related U.S. Application Data
(60) Provisional application No. 60/587,130, filed on Jul. 12, 2004.
(60) Provisional application No. 60/611,438, filed on Sep. 20, 2004.

(51) Int. Cl. A61F 5/02

(52) U.S. Cl. 2/44

(57) ABSTRACT

A full body safety harness to be worn by a person includes an upper torso portion and a lower seat portion. The upper torso portion is operatively connected to the lower seat portion by a first connector on a first lateral side and a second connector on a second lateral side thereof. The first connector and the second connector enable forward and rearward rotation of the upper torso portion relative to the lower seat portion (as in the case of forward and rearward bending by a user of the harness) without causing a significant increase in tension in the lower seat portion. A safety harness to be worn by a person which includes at least one strap section including a cam buckle in operative connection therewith to adjust a fit of the strap section. A safety harness includes a label pack system including a base having an attachment mechanism to attach the base to a strap of the safety harness and a closure in operative connection with the base. The base and the enclosure at least partially enclose at least one label (and more typically a plurality of labels) when the closure in a closed state. A safety harness includes a strap section having an interior surface that is adjacent a wearer when the safety harness is worn and an exterior surface generally opposite the interior surface. The interior surface is perceptibly different from the exterior surface so that a wearer can distinguish the interior surface from the exterior surface. A safety harness includes at least one strap section have a first end; the first end including an end member in operative connection therewith, the end member includes a retaining member to connect the end member to the strap or to another strap of the harness.

### CROSS-REFERENCE TO RELATED APPLICATIONS

The present invention claims benefit of U.S. Provisional Patent Application Ser. No. 60/587,130, filed Jul. 12, 2004, and U.S. Provisional Patent Application Ser. No. 60/611,438, filed Sep. 20, 2004, the disclosures of which are incorporated herein by reference.

### BACKGROUND OF THE INVENTION

The present invention relates generally to full body safety harnesses, and particularly to safety harnesses which, for example, provide improved or enhanced range of motion, comfort, ease of donning, ease of adjustment and ease of adding accessory equipment as compared to other safety harnesses.

References set forth herein may facilitate understanding of the present invention or the background of the present invention. Inclusion of a reference herein, however, is not intended to and does not constitute an admission that the reference is available as prior art with respect to the present invention.

Safety harnesses are commonly used as part of a fall protection system for persons subjected to the potential of a fall from a height. In the workplace, full-body safety harnesses are required when working at a height of six feet or greater. Such harnesses, which typically include both an upper torso portion (having, for example, shoulder straps) and a lower torso or seat portion (having, for example one or more leg straps and sometimes a seat strap), can be designed in many alternative manners.

Many currently available full-body safety harnesses are manufactured from relatively inelastic, woven webbing materials such as nylon or polyester. A flexible and elastic harness, as described in U.S. Pat. No. 6,006,700, assigned to the assignee of the present invention, the disclosure of which is incorporated herein by reference, has been introduced that greatly improves the comfort of the user during normal use of the safety harness. A safety harness with blunted edges for further increasing the comfort of the user is disclosed in U.S. Pat. No. 6,739,427, assigned to the assignee of the present invention, the disclosure of which is incorporated herein by reference.

Although the comfort of safety harnesses during normal use and even during a fall arrest has been greatly improved in the above-described harnesses, the underlying design of currently available safety harnesses still leads to a number of problems including, for example, range of motion problems, comfort problems, donning problems, adjustment problems and webbing wear problems. FIG. 1 illustrates an embodiment of a conventional, commercially available full-body safety harness 10 Safety harness 10 includes an upper torso portion 12 comprising first and second shoulder straps 20 and 30, respectively, for extending over the shoulders of the user and a multi-component chest strap 40 for extending over a portion of the chest of the user.

A first end of each of shoulder straps 20 and 30 extends down over the back of the user to form first and second generally longitudinal back straps 22 and 32, respectively. Longitudinal back straps 22 and 32 of shoulder straps 20 and 30 cross through and connect to a typical D-ring 50 as known in the art. D-ring 50 includes a harness connection portion 52 and an anchor portion 54. Harness connection portion 52 enables fastening of D-ring 50 to safety harness 10 via longitudinal back straps 22 and 32. Anchor portion 54 is adapted to be connected to a nylon rope, a chain, webbing or other connector which may be used to anchor the person wearing safety harness 10.

After crossing and passing through D-ring 50, shoulder straps 20 and 30 are connected via a generally latitudinal back strap 60. Latitudinal back strap 60 passes generally latitudinally over a portion of the back of the user.

A second end of each of shoulder straps 20 and 30 extends downward over the front of the user to from generally longitudinal first and second front straps 24 and 34, respectively. A first chest strap portion 42 is attached to front strap 24 and a second chest strap portion 44 is attached to front strap 34. Each of first and second chest straps 42 and 44 have cooperating fastening members 46 and 48 on the ends thereof to enable attachment of first and second chest straps 42 and 44 to form chest strap 40. As known in the art, first and second chest straps 42 and 44, respectively, are preferably attached via an adjustable mating friction buckle mechanism, including, for example, cooperating fastening members 46 and 48.

First and second front straps 24 and 34 of shoulder straps 20 and 30, respectively, extend further downward and preferably include adjustment members 26 and 36 (for example, adjustable friction buckles) as known in the art for adjustment of the fit of safety harness 10 on the upper torso of the user. Extending still further downward, extensions 24a and 34a of first and second front straps 24 and 34 converge and, in connection with several other components of safety harness 10 as described below, form a lower torso, seat or subpelvic portion 70. First and second front extension straps 24a and 34a connect at section 98, passing to the rear and under the seat of the user.

Attached to and extending from seat portion 70 are a first and a second leg strap 80 and 90, respectively. Each of first and second leg straps 80 and 90 pass around the upper leg of the user to be attached to the distal end of first and second longitudinal back straps 22 and 32, respectively. The distal ends of each of first and second leg straps 80 and 90 and the distal ends of each of longitudinal back straps 22 and 32 thus preferably comprise cooperating fastening members (82 and 92 and 28 and 38, respectively) such as adjusting buckle members as known in the art.

U.S. Pat. No. 5,957,091 discloses another design of a body harness including a pair of shoulder straps, a pair of leg straps and a pair of rigid hip plates. The shoulder straps are connected at both ends to the hip plates and pass through a back pad and through a shoulder strap retainer (preferably a front or a chest strap). Each leg strap is connected at one end to one of the same rigid hip plates to which the shoulder straps are connected and can be connected at the other end to one of the same rigid hip plates to which the shoulder straps are connected. Adjustment of the fit of the shoulder straps of the body harness of U.S. Pat. No. 5,957,091 is accomplished through the use of a set of three adjacent parallel slots in the rigid hip plates. Adjustment of the leg straps of the body harness of U.S. Pat. No. 5,957,091 is accomplished through the use of a quick fit buckle.

In general, it is difficult, for example, to bend forward or rearward in a harness such as harness 10 and other harnesses because of the resistance of the interconnected strapping material forming the harness. In that regard, a continuous strap or a series of interconnected strap sections from part of the upper torso portion as well as the lower seat section. Use of an elastic strapping material as disclosed in U.S. Pat. No. 6,006,700 facilitates such bending, but resistance is still present. In that regard, bending forward and/or rearward causes significant pulling (that is, tension in the strapping) on the lower torso or seat portion of the harness. Moreover, the interconnected, crossing nature of currently available harnesses such as harness 10 causes adjustment of the fit of one portion of harness 10 to affect the fit/tension in other portions of the harness. For example, adjustment of buckle 26 to adjust the length of shoulder strap 20 affect the fit/tension of one or both of leg straps 80 and 90.

In addition to the above-identified problems, users of currently available safety harnesses find it very difficult to adjust the fit of the safety harness and other fall protection safety gear using friction buckles and other types of adjustment mechanisms found on such safety harnesses. Moreover, this difficulty can often be increased with the use of harness webbing material designed to increase the comfort of the user. Furthermore, it is often difficult to don currently available safety harnesses. Although efforts have been made to simplify the donning of a safety harness by, for example, fabricating the harness material so that the harness maintains its form when not worn as described in U.S. Pat. No. 6,739,427 or by providing a back connector assembly sufficiently rigid to give an undonned harness some shape (see, for example, U.S. Pat. No. 6,253,874), problems in donning persist. For example, it is often difficult of the user of the harness to determine which side of the harness material is the outer or inner side, leading to donning of the harness in and inside-out conformation. Moreover, webbing material used in currently available safety harnesses and other fall protection safety gear is susceptible to fraying, particularly at the end thereof.

It is very desirable to develop improved safety harnesses that reduce or eliminate the above and other problems with currently available harnesses.

### SUMMARY OF THE INVENTION

In one aspect, the present invention provides a full body safety harness to be worn by a person including an upper torso portion and a lower seat portion. The upper torso portion is operatively connected to the lower seat portion by a first connector on a first lateral side and a second connector on a second lateral side thereof. The first connector and the second connector enable forward and rearward rotation of the upper torso portion relative to the lower seat portion (as in the case of forward and rearward bending by a user of the harness) without causing a significant increase in tension in the lower seat portion. The less tension increases in the lower torso portion during bending, the better. Such tension increases in the lower torso portion of the harnesses of the present invention upon bending can, for example, be reduced to less than 10% or even substantially eliminated.

In one embodiment, at least one of the first connector and the second connector includes a rotating joint. Both of the first connector and the second connector can include a rotating joint. In another embodiment, at least one of the first connector and the second connector includes a ring member about which at least one strap of the upper torso portion or the lower seat portion is slidably attached. For example, the first connector can include an upper ring member and a lower ring member, wherein at least one end of a shoulder strap of the upper torso portion is slidably attached to the upper ring member, and at least one strap of leg strap of the lower seat portion is slidably attached to the lower ring member.

The upper torso portion can, for example, include a first shoulder strap adapted to pass over a first shoulder of the person, wherein a first end of the first shoulder strap is adapted to extend over the front of the person to connect to the first connector and a second end of the first shoulder strap is adapted to extend over the back of the person to connect to the second connector. The upper torso portion can further include a second shoulder strap adapted to pass over a second shoulder of the person, wherein a first end of the second shoulder strap is adapted to extend over the front of the person to connect to the second connector and a second end of the second shoulder strap is adapted to extend over the back of the person to connect to the first connector. The safety harness can further include a first leg strap connected to the first connector and a second leg strap connected to the second connector. In one embodiment at least one of the first shoulder strap and the second shoulder strap includes an adjustment mechanism to adjust the length thereof and at least one of the first leg strap and the second leg strap includes an adjustment mechanism to adjust the length thereof. The first shoulder strap can include a first shoulder strap adjustment mechanism to adjust the length of the first shoulder strap, and the second shoulder strap can include a second adjustment mechanism to adjust the length of the second shoulder strap. The first leg strap can include a first leg strap adjustment mechanism to adjust the length of the first leg strap, and the second leg strap can include a second leg strap adjustment mechanism to adjust the length of the second leg strap.

In another aspect, the present invention provides a full body safety harness to be worn by a person including an upper torso portion and a lower seat portion, wherein the upper torso portion is operatively connected to the lower seat by a first rotating connector on a first lateral side and a second rotating connector on a second side thereof. The first rotating connector and the second rotating connector enable rotation of the upper torso portion relative to the lower seat portion (as occurs, for example, upon forward and rearward bending by the person).

The first rotating connector can be positioned to rotate about a point in the vicinity of the axis of rotation of the hips of the person when worn by the person. Likewise, the second rotating connector can be positioned to rotate about a point in the vicinity of the axis of rotation of the hips of the person when worn by the person. In one embodiment, the first rotating connector (and/or the second rotating connector) includes an upper connective member and a lower connective member. The upper connective member is rotatably connected to the lower connective member. The upper connective member can, for example, be connected to the lower connective member by a shaft about which the upper connective member can rotate relative to the lower connective member. The upper connective member can include at least one attachment adapted to connect to harness strapping, and the lower connective member can include at least one attachment adapted to connect to harness strapping. In one embodiment, the upper connective member includes at least two slots formed therein for attachment of harness strapping, and the lower connective member includes at least two slots formed therein of attachment of harness strapping.

The rotating connectors of the present invention can further include an accessory connector attached to an outer surface thereof which is adapted to connect accessories to the full body safety harness. Likewise, the rotating connectors of the present invention can include a belt connector attached to an inner surface thereof which is adapted to connect the full body safety harness to a belt (for example, a safety positioning belt comprising an anchor attachment or a tool belt).

The upper torso portion of the full body safety harness can include a first shoulder strap adapted to pass over a first shoulder of the person, wherein a first end of the first shoulder strap is adapted to extend over the front of the person to connect to the first rotating connector, and a second end of the first shoulder strap is adapted to extend over the back of the person to connect to the second rotating connector. The upper torso portion can further include a second shoulder strap adapted to pass over a second shoulder of the person, wherein a first end of the second shoulder strap is adapted to extend over the front of the person to connect to the second rotating connector, and a second end of the second shoulder strap is adapted to extend over the back of the person to connect to the first rotating connector. In this embodiment, the first shoulder strap and the second shoulder strap cross over each other in the back of the full body safety harness. The lower torso portion can include a first leg strap connected to the first rotating connector and a second leg strap connected to the second rotating connector. At least one of the first shoulder strap and the second shoulder strap can include an adjustment mechanism to adjust the length thereof and at least one of the first leg strap and the second leg strap can include an adjustment mechanism to adjust the length thereof. In one embodiment, the first shoulder strap includes a first shoulder strap adjustment mechanism to adjust the length of the first shoulder strap; the second shoulder strap includes a second adjustment mechanism to adjust the length of the second shoulder strap; the first leg strap include a first leg strap adjustment mechanism to adjust the length of the first leg strap; and the second leg strap includes a second leg strap adjustment mechanism to adjust the length of the second leg strap.

In one embodiment, the full body safety harness of further includes a cam buckle in operative connection with at least one of strap section of the upper torso portion or the lower torso portion to adjust a fit of the strap section. The cam buckle can include a base and a locking member in moveable, operative connection with the base. The locking member can include an abutment surface moveable into and out of contact with the strap. In one embodiment, the locking member is rotatably attached to the base, and the abutment surface is biased in connection with the strap.

The base can include a strap support over which a strap of the safety harness passes. The abutment surface of the locking member can be biased in connection with a first surface of the strap over a section of the strap wherein a second, opposing surface of the strap contacts the support member. The locking member can include a lever arm to which force is applied to move the locking member into a release position in which the abutment surface is out of contact with the strap. The cam buckle can also include an activating mechanism that must be activated to enable the locking member to be moved to a release position.

In another embodiment, the safety harness includes a connector attached to at least one strap section of the upper torso portion or the lower torso portion. The connector includes a first attachment mechanism to attach the connector to the strap and a second attachment mechanism to attach an item to the connector. The connector can, for example, include a base, and the first attachment mechanism can includes a pair of slots formed in the base. The connector can further include a closure in operative connection with the base. In one embodiment, the base and the enclosure at least partially enclose at least one label (including, for example, printed information about or relative to the harness) when the closure in a closed state. In one embodiment, the closure includes a hinge about which the closure is rotatable to the closed state and to an open state. For example, the at least one label can be accessible when the closure is in an open state. The closure can further include a releasable locking mechanism to releasably cooperate with the base to hold the closure in a closed state.

In another embodiment, the safety harness further includes at least one strap section having an interior surface that is adjacent a wearer when the safety harness is worn and an exterior surface generally opposite the interior surface. The interior surface is perceptibly different from the exterior surface so that a wearer can distinguish the interior surface from the exterior surface. The interior surface can, for example, have a different color from the exterior surface. The interior surface can, for example, have a different texture from the exterior surface. The interior surface can, for example, be softer than the exterior surface. The exterior surface can, for example, have a greater abrasion resistance than the interior surface. In one embodiment, the interior surface and the exterior surface are formed about a generally tubular outer shell. In this embodiment, he strap section can further include a flexible inner material.

In a further embodiment, the safety harness further includes at least one strap section have a first end. The first end includes an end member in operative connection therewith. The end member includes a retaining member to connect the end member to the at least one strap or to another strap of the harness. The retaining member can, for example, include an extending arm to extend around the another strap. In one embodiment, the extending member is biased against the another strap when extending around the another strap.

In another aspect, the present invention provides a full body safety harness to be worn by a person including an upper torso portion and a lower seat portion. The upper torso portion is operatively connected to the lower seat by at least one connector adapted to enable rotation of the upper torso portion over a range of positions forward and rearward relative to the lower seat portion without causing significant tension in the lower seat portion.

In a further aspect, the present invention provides a full body safety harness to be worn by a person including an upper torso portion and a lower seat portion. The upper torso portion is removably connected to the lower torso or seat portion via a first connector on a first lateral side of the harness and a second connector on a second lateral side of the harness.

In one embodiment, the first connector includes an first upper connecting member to which at least one strap of the upper torso portion is connected and a first lower connecting member to which at least one strap of the lower torso portion is connected. The first upper connecting member and the first lower connecting member are adapted to be placed in operative connection. The second connector includes a second upper connecting member to which at least one strap of the upper torso portion is connected and a second lower connecting member to which at least one strap of the lower torso portion is connected. The second upper connecting member and the second lower connecting member are adapted to be placed in operative connection.

The first upper connecting member and the first lower connecting member in one embodiment are adapted to be placed in operative connection such that the first upper connecting member is rotatable about an axis relative to the first lower connecting member. The second upper connecting member and the second lower connecting member can also be adapted to be placed in operative connection such that the second upper connecting member is rotatable about an axis relative to the second lower connecting member.

In still a further aspect, the present invention provides a method of fabricating a full body safety harness, including: forming an upper torso portion of the full body safety harness; forming separately from the upper torso portion a lower seat portion; and connecting the upper torso portion to the lower seat portion via a first connector on a first lateral side of the full body safety harness and a second connector on a second side of the full body safety harness. In one embodiment, the first connector includes an first upper connecting member to which at least one strap of the upper torso portion is connected and a first lower connecting member to which at least one strap of the lower seat portion is connected. The first upper connecting member and the first lower connecting member are adapted to be placed in operative connection. The second connector can also includes a second upper connecting member to which at least one strap of the upper torso portion is connected and a second lower connecting member to which at least one strap of the lower seat portion is connected. The second upper connecting member and the second lower connecting member are adapted to be placed in operative connection.

The method can further include: forming a plurality of upper torso portions, each have a unique configuration; and selecting one of the plurality of upper torso portions to be connected to the lower seat portion. Likewise, the method can further include: forming a plurality of lower seat portions, each have a unique configuration; and selecting one of the plurality of lower seat portions to be connected to the upper torso portion.

In another aspect, the present invention provides a safety harness to be worn by a person which includes at least one strap section. The strap section includes a cam buckle in operative connection therewith to adjust a fit of the strap section. The can buckle can, for example, include a base and a locking member in moveable, operative connection with the base. The locking member includes an abutment surface moveable into and out of contact with the strap. The locking member can be rotatably attached to the base. The abutment surface can be biased in connection with the strap.

The base can include a strap support over which the strap passes. The abutment surface of the locking member can be biased in connection with a first surface of the strap over a section of the strap wherein a second, opposing surface of the strap contacts the support member. In one embodiment, the locking member includes a lever arm to which force is applied to move the locking member into a release position in which the abutment surface is out of contact with the strap. The cam buckle can also an activating or actuating mechanism that must be activated to enable the locking member to be moved to a release position. Requiring such a dual action to cause a release of the strap section can help prevent accidental release.

In another aspect, the present invention provides a connector for use in connection with a strap of a safety harness. The connector includes a first attachment mechanism to attach the connector to the strap and a second attachment mechanism to attach an item to the connector. In one embodiment, the connector includes a base, and the first attachment mechanism includes a pair of slots formed in the base. In one embodiment, the connector includes a closure in operative connection with the base, and the base and the enclosure at least partially enclose at least one label when the closure in a closed state.

In a further aspect, the present invention provides a label pack system for use in connection with a safety harness. The label pack system includes a base having an attachment mechanism to attach the base to the strap of the safety harness and a closure in operative connection with the base. The base and the enclosure at least partially enclose at least one label (and more typically a plurality of labels) when the closure in a closed state. The labels include, for example, textual and/or graphical information about the safety harness.

The closure can include a hinge about which the closure is rotatable to the closed state and to an open state. The label(s) are accessible when the closure is in an open state. The closure can, for example, include a releasable locking mechanism to releasably cooperate with the base to hold the closure in a closed state.

In another aspect, the present invention provides a safety harness including at least one strap section having a first end; wherein the first end has an end member in operative connection therewith. The end member includes a housing having a seating therein. The end member further includes a clip member having a first extending arm and a second extending arm in between which the first end of the strap is held. The clip is seated within the seating of the housing so that the first extending arm and the second extending arm are forced into contact with first end of the strap. In one embodiment, each of the first extending arm and the second extending arm include teeth that contact the first end of the strap. The clip member can further include a locking member that cooperates with the housing to hold the clip member in operative connection with the seating of the housing. The first extending arm and the second extending arm of the clip can, for example, be formed form an integral piece of resilient material. In one embodiment, the housing includes a retaining arm to connect the end member to the strap or to another strap of the harness.

In still a further aspect, the present invention provides a safety harness including a strap section having an interior surface that is adjacent a wearer when the safety harness is worn and an exterior surface generally opposite the interior surface. The interior surface is perceptibly different from the exterior surface so that a wearer can distinguish the interior surface from the exterior surface. In one embodiment, the interior surface has a color different from a color of the exterior surface. The interior surface can also or alternatively have a texture different from a texture of the exterior surface. The interior surface can, for example, be softer than the exterior surface to provide comfort to the wearer. The exterior surface can be of a more durable or rugged material (for example, having greater abrasion resistance than the interior surface).

In one embodiment, the interior surface and the exterior surface can form or be formed about on a generally tubular outer shell. In this embodiment, the strap section can further include a flexible inner material. A wear indicator material can be placed between the outer shell and the inner material such that any opening in the outer shell causes the wear indicator material to be visible.

In another aspect, the present invention provides a safety harness including at least one strap section have a first end. The first end includes an end member in operative connection therewith. The end member includes a retaining member to connect the end member to the strap or to another strap of the harness. The retaining member can, for example, include an extending arm adapted to extend around strap or another strap. In one embodiment, the extending member is adapted to be biased against the strap or against the another strap when extending around the strap or the another strap.

### BRIEF DESCRIPTION OF THE DRAWINGS

FIG. 1 illustrates a commercially available full body safety harness.

FIG. 2A illustrates a front perspective view of a person wearing an embodiment of a full body safety harness of the present invention, wherein the person is standing erect.

FIG. 2B illustrates a rear perspective view of a person wearing the full body safety harness of FIG. 2A, wherein the person is standing erect.

FIG. 2C illustrates a side view of a person wearing the full body safety harness of FIG. 2A, wherein the person is standing erect.

FIG. 2D illustrates a side view of a person wearing the full body safety harness of FIG. 2A, wherein the person is bending forward in a crouched position.

FIG. 2E illustrates an enlarged, prospective view of an embodiment of a rotating connector of the full body safety harness of FIG. 2A.

FIG. 2F is an exploded, perspective view of connector of FIG. 2E.

FIG. 2G is a side, cross-sectional view of the connector of FIG. 2E

FIG. 3A illustrates a side view of another embodiment of a rotating connector of the present invention.

FIG. 3B illustrates a side view of the rotating connector of FIG. 3A.

FIG. 3C illustrates a side, cross-sectional view of the rotating connector of FIG. 3A.

FIG. 3D illustrates a perspective, exploded view of the rotating connector of FIG. 3A.

FIG. 4A illustrates another embodiment of a connector of the present invention.

FIG. 4B illustrates another embodiment of a connector of the present invention.

FIG. 4C illustrates another embodiment of a connector of the present invention.

FIG. 5 illustrates the attachment of an accessory to the rotating connector of FIG. 2A.

FIG. 6A illustrates one embodiment of a full body safety harness of the present invention including a first upper torso module and a first lower seat module.

FIG. 6B illustrates another embodiment of a full body safety harness of the present invention including the first upper torso module illustrated in FIG. 6A and a second lower seat module, different in configuration from the first lower seat module.

FIG. 6C illustrates the use of the first upper torso module of FIG. 6A with a tool belt and without a lower seat module.

FIG. 7 illustrates another embodiment of a rotating connector of the present invention adapted to enable ready disconnection of the upper torso portion or module of the harness from the lower seat portion or module of the harness.

FIG. 8A illustrates a top plan view of an embodiment of a buckle of the present invention.

FIG. 8B illustrates a rear view of the buckle of FIG. 8A.

FIG. 8C illustrates a side, cross-sectional view of the buckle of FIG. 8A.

FIG. 8D illustrates a side view of the buckle of FIG. 8A.

FIG. 8E illustrates a perspective exploded or disconnected view of the buckle of FIG. 8A.

FIG. 8F illustrates a side, cross-sectional view of the buckle of FIG. 8A in operative connection with a harness strap in which the locking member in a release state.

FIG. 8G illustrates a side, cross-sectional view of the buckle of FIG. 8A in operative connection with a harness strap in which the locking member in a locking state.

FIG. 9A of the present invention illustrates a perspective view of an embodiment of a connector of the present invention and a label pack system in operative connection therewith in which a closure of the label pack system is in an open state.

FIG. 9B illustrates a perspective view of the connector of FIG. 9A in which the closure is in a closed state.

FIG. 9C illustrates a perspective view of the base of the connector of FIG. 9A.

FIG. 10A illustrates a perspective view of a strap end member in an exploded or disconnected state.

FIG. 10B illustrates a perspective view of the strap end member of FIG. 10A in a connected or assembled state.

FIG. 10C illustrates a perspective view of another embodiment of a strap end member in an exploded or disconnected state.

FIG. 11 illustrates a perspective view of an embodiment of a strap section of the present invention having an interior surface and an exterior surface that are perceptibly different.

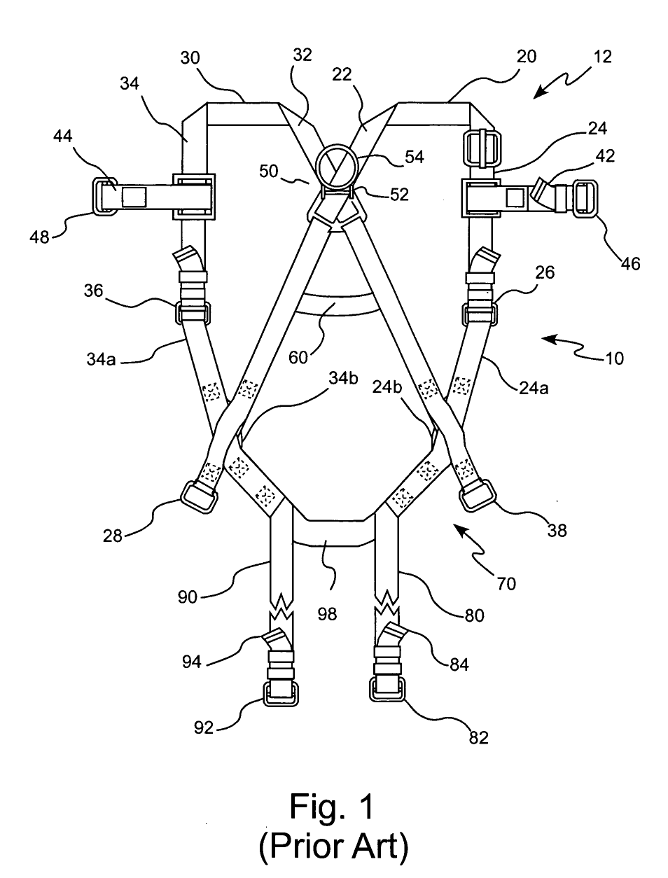

### DETAILED DESCRIPTION OF THE INVENTION

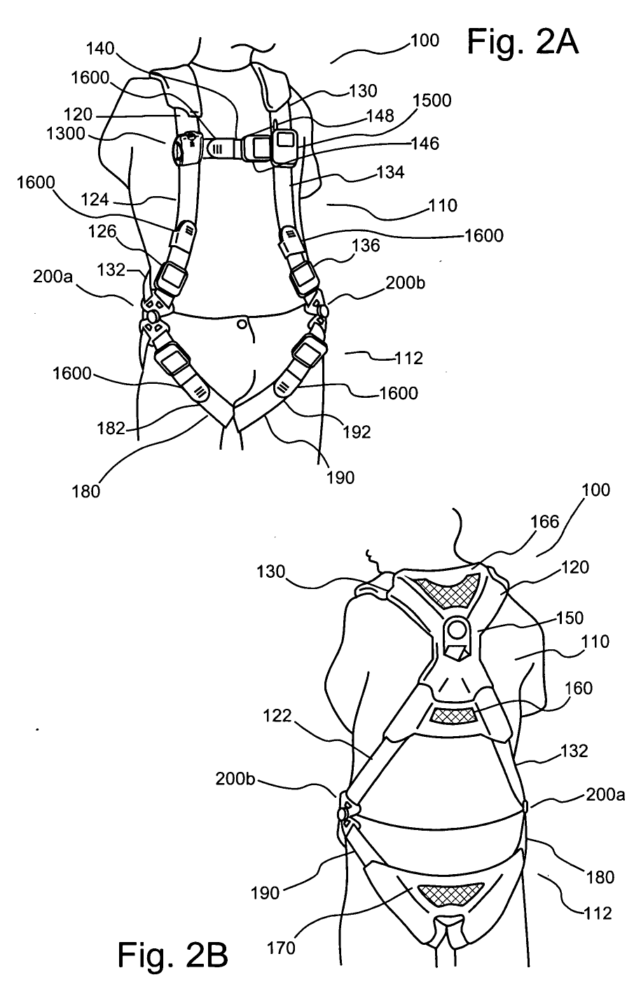

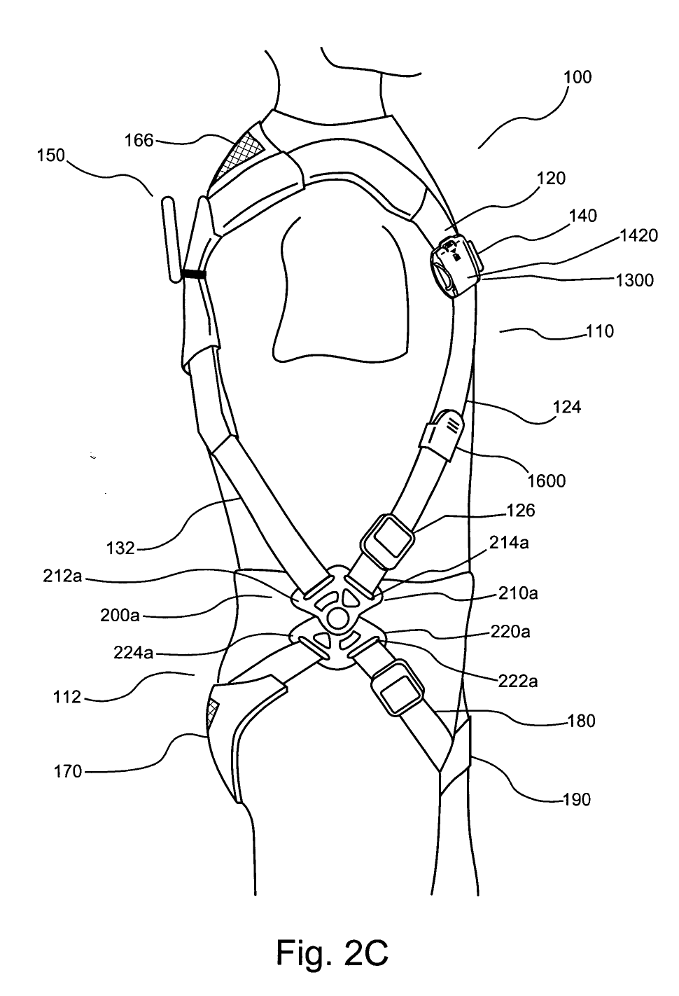

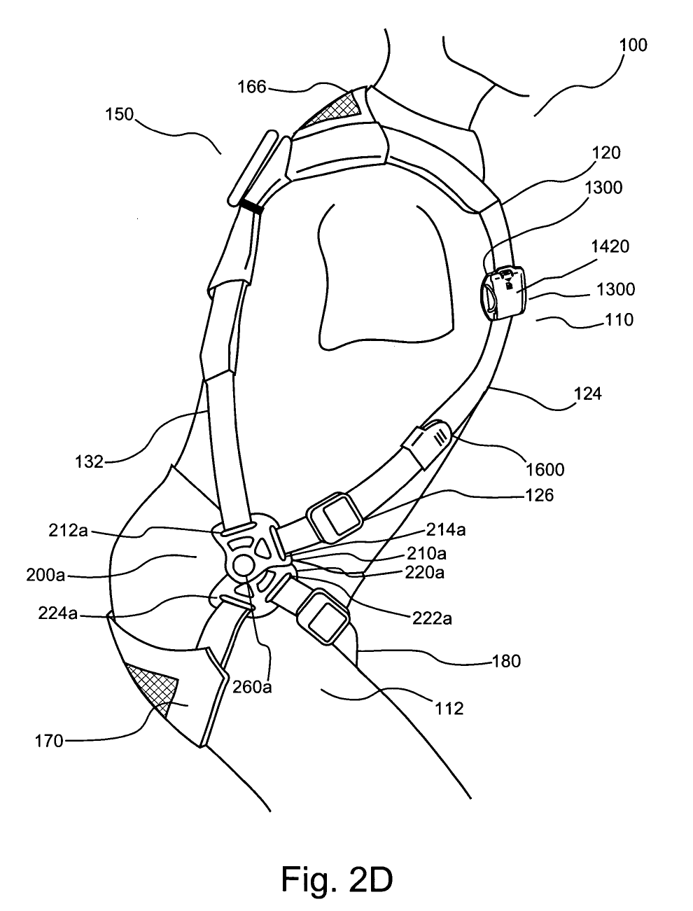

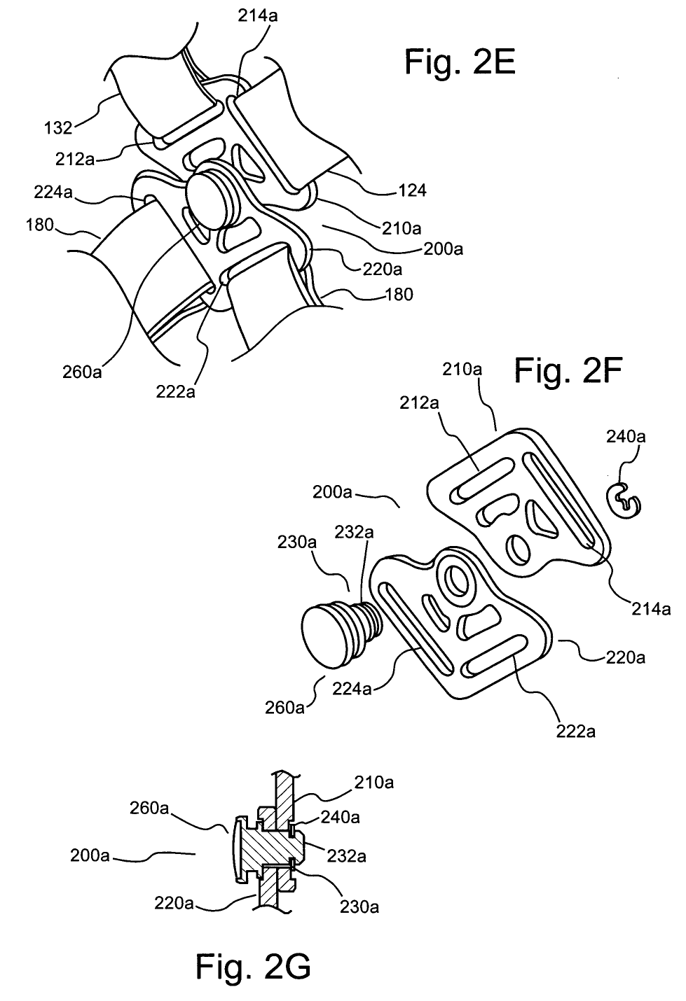

FIGS. 2A through 2G illustrate one embodiment of a full body safety harness 100 of the present invention. Safety harness 100 includes an upper torso section, portion or module 110 and a lower torso (seat) section, portion or module 112. Upper torso portion 110 includes a first shoulder strap 120 and a second shoulder strap 130 extending over the shoulders of the user and a multi-component chest strap 140 extending between first shoulder strap 120 and second shoulder strap 130. First ends of each of shoulder straps 120 and 130 extend down over the back of the user to form first and second generally longitudinal back straps 122 and 132, respectively. Back straps 122 and 132 cross through and connect to a D-ring 150 in a manner similar to that described above in connection with D-ring 50. After crossing and passing through D-ring 150, back strap sections 122 and 132 of shoulder straps 120 and 130 are connected via a generally latitudinal back strap 160, which passes generally latitudinally over a portion of the back of the user. In the illustrated embodiment, back straps 122 and 132 also cross through a back pad 166 in the region of D-ring 150 which, among, other functions can assist in maintaining a desired distance from neck of the user to D-ring 150. Back straps 122 and 132 are attached at their respective ends to connectors 200b and 200a, respectively, that operate to connect upper torso portion or module 110 to lower seat portion or module 112. In that regard back straps 122 and 132 can, for example, be looped around slots 212b and 212a formed in upper connecting members 210b and 210a of connectors 200b and 200a, respectively, (see, for example, FIGS. 2C through 2E) and stitched.

A second end of each of shoulder straps 120 and 130 extends downward over the front of the user to form generally longitudinal first and second front straps 124 and 134, respectively. Chest strap 140 is attached between front strap 124 and front strap 134 of shoulder straps 120 and 130, respectively. At least one fastening member 146 can be provide to enable opening/disconnection of chest strap 140. One or more adjustment mechanisms 148 (for example, adjustable cam buckle mechanisms as discussed further below) can be provided to adjust the length of chest strap 140. In an alternative embodiment, as known in the art, first and second front straps 124 and 134 can cross through a front pad, as known in the art, and chest strap 140 can be eliminated.

First and second front straps 124 and 134 extend further downward from chest strap 140 and can include adjustment members 126 and 136 (for example, adjustable cam buckles as discussed further below) for adjustment of the fit of safety harness 100 on the upper torso of the user. Front straps 124 and 134 are connected at the ends thereof to connectors 200a and 200b, respectively. In that regard front straps 124 and 134 can, for example, be looped through slots 214a and 214b formed in upper connecting members 210a and 210b of connectors 200a and 200b (see, for example, FIGS. 2C through 2E) and stitched.

Lower torso or seat portion 112 of safety harness 100 can, for example, include leg straps 180 and 190. Leg strap 180 is attached to connector 200a at a first or front end thereof via slot 222a formed in a lower connecting member 220a of connector 200a (see, for example, FIGS. 2C through 2E). A second or rearward end of leg strap 180 is attached to lower connecting member 220a via slot 224a. Similarly, leg strap 190 is attached to connector 200b at a first or front end thereof via slot 222b formed in a lower connecting member 220b of connector 200b. A second or rearward end of leg strap 190 is attached to lower connecting member 220b via slot 224b. As illustrated, for example in FIG. 2A, leg straps 180 and 190 can include adjustment members or mechanisms 182 and 192, respectively, (for example, adjustable cam buckles as discussed further below) for adjustment of the fit of leg straps 180 and 190. A seat 170 can be attached to and extends between leg straps 180 and 190. Seat 170 can, for example, be fabricated from a mesh material or from a strapping material.

In the embodiment of FIGS. 2A through 2G, connectors 200a and 200b are identical in design and operation. The design and operation of connectors 200a and 200b is discussed herein with reference to connector 200a. In that regard, upper connecting member 210a and lower connecting member 220a (which can each, for example, be formed separately from an integral or monolithic piece metal such as aluminum) are rotatably or pivotably connected via a shaft 230a (see, for example, FIGS. 2F and 2G), which is maintained in operative connection with connecting members 210a and 220a via a locking pin 240a which seats in a seating or groove 232a formed in shaft 230a. As clear to one skilled in the art, connectors 200a and 200b are preferably fabricated from materials (which can, for example, include one or more metals, high-strength polymeric materials, high-strength composite materials or combinations thereof) of sufficient strength and are preferably connected in such a manner such that connectors 200a and 200b can withstand the loads experienced in a fall. Like the strapping of safety harness 100, the materials and construction of connectors 200a and 200b can, for example, provide an ultimate tensile load of at least 4000 pounds or at least 5000 pounds (as discussed, for example, for harness components generally in U.S. Pat. Nos. 6,006,700 and 6,739,427).

As illustrated in a comparison of FIGS. 2C and 2D, rearward or forward bending of the user results in a rotation of upper connecting member 210a relative to lower connecting member 220a. Unlike the case of harness 10, the relative rotation of upper connecting member 210a and lower connecting member 220a of connector 200a (and the relative rotation of upper connecting member 210b and lower connecting member 220b of connector 200b) substantially prevents tension in upper torso portion 110 and lower seat portion 112 during such bending. In that regard, connectors 200a and 200b effectively “decouple” the strains experienced in the straps of upper torso portion 110 from the strains experienced in the straps of lower seat portion 112 during forward and rearward bending of the user. The range and freedom of movement of a user of safety harness 100 is thereby substantially enhanced as compared to other safety harnesses. Moreover, connectors 200a and 200b also prevent gapping of the straps away from the body in upper torso portion 110, which is often experienced in currently available harnesses upon forward or rearward bending. Such gapping in other safety harnesses can present a safety concern as gapped strapping can catch on objects when the safety harness is in use. Still further, connectors 200a and 200b enable the adjustment of strap length, tension and/or fit in upper torso portion 110 without substantially affecting the strap length, tension and/or fit in lower seat portion 112, thereby enhancing the comfort of the fit of safety harness 100 as compared to other safety harnesses. Indeed, such connectors enable independent adjustment of straps in upper torso portion 110 and lower seat portion 120 using adjustment mechanisms or members in each of upper torso portion 100 and lower seat portion 120. In that regard, tightening of shoulder strap 120 via adjustment mechanism 126 may cause some increased tension in leg strap 180 by pulling upward on connector 200a. However, the fit of leg strap 180 can be adjusted via adjustment mechanism 182.

In general, the benefits afforded by connectors 200a and 200b are realized regardless of the material chosen for the straps. In that regard, relatively inelastic, woven webbing materials such as nylon or polyester commonly used in many currently available are well suited for use in the present invention. Moreover, the material described in U.S. Pat. No. 6,006,700 and/or U.S. Pat. No. 6,739,427 can also be used. Such materials are, for example, currently used in the DURAFLEX® and the DURAFLEX PYTHON® harnesses currently available from Bacou-Dalloz Fall Protection of Franklin, Pa.

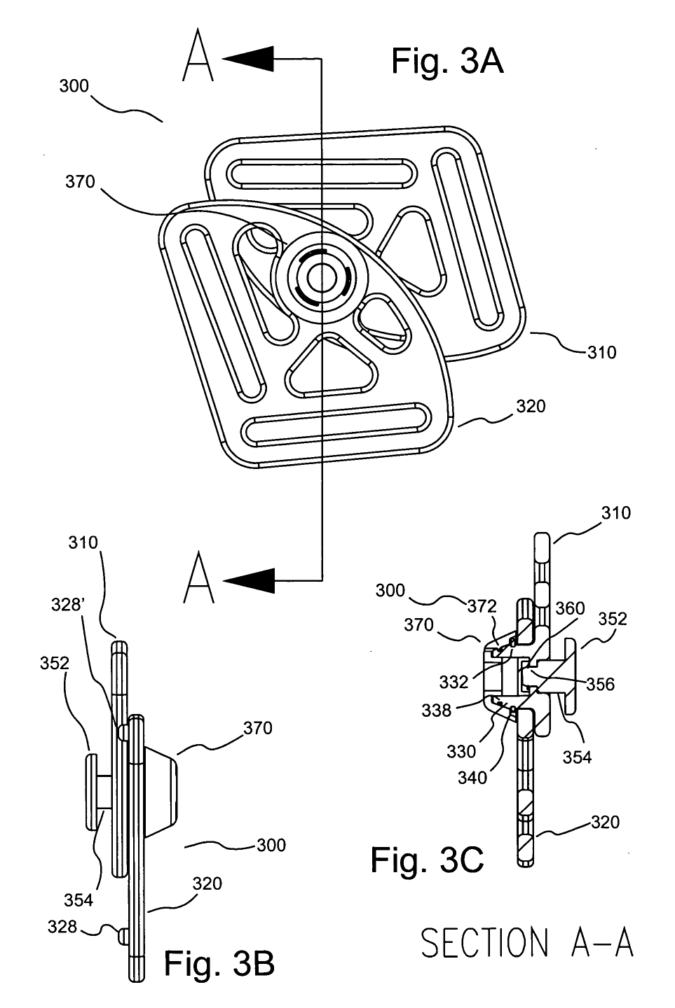

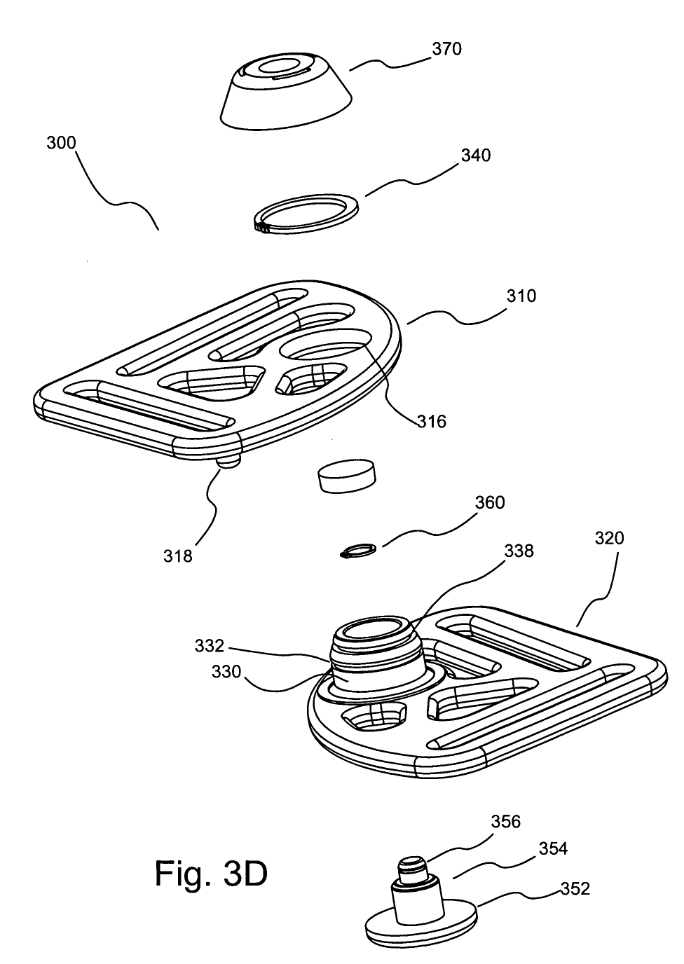

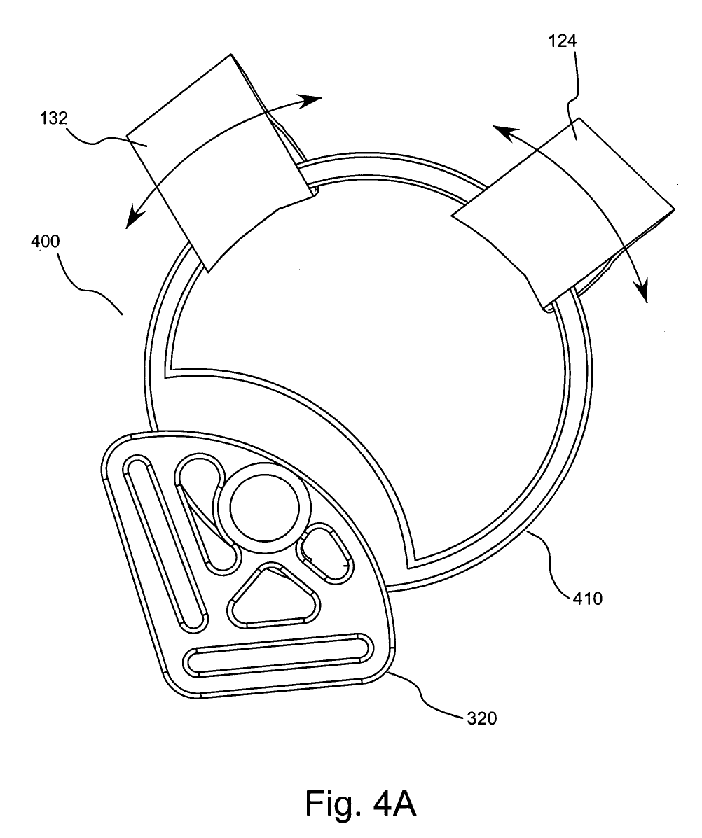

FIGS. 3A through 3D illustrate another embodiment of a connector 300 for use in the present invention to operatively connect an upper torso portion of a safety harness to a lower torso or seat portion of the safety harness. Connector 300 operates similarly in many respects to connectors 200a and 200b as described above. In that regard, connector 300 includes an upper connecting member 310 and a lower connecting member 320 which are rotatably connected via a shaft 330 which projects from upper connecting member 310. Lower connecting member 320 includes a generally circular passage 316 through which shaft 330 projects. Upper connecting member 310 and lower connecting member 320 are maintained in rotatable connection (about shaft 330) via a locking ring 340 which seats within a seating or groove 332 formed in shaft 330. As illustrated in the embodiment of FIGS. 3A through 3D, lower connecting member 320 can include abutment members 328 and 328′ which abut upper connecting member 310 to limit the range of relative rotation between upper connecting member 310 and lower connecting member 320 to, for example, prevent upper connecting member 310 from contacting the leg straps of the lower torso portion and causing excessive wear therein. The range of motion of upper connecting member 310 relative to lower connecting member 320 can, for example, be limited to approximately 30° to approximately 40° to prevent such wear while still providing an ample range of motion for bending of the user without causing significant tension in the straps of the lower seat portion.

Connector 300 further includes an inward attachment or connector 350 which includes a flange 352 on an inward projecting end of a shaft 354 thereof to which, for example, a belt such as a positioning safety belt or a tool belt can be attached to connector 300. As clear to one skilled in the art, other attachment mechanisms as known in the art are suitable for use in attaching belts to the connectors of the present invention. As known in the art, safety positioning belts can, for example, include an anchor attachment such as a D-ring as known in the art to connect a lifeline or lanyard thereto. Attachment 350 is maintained in operative connection with upper connecting member 310 by a locking ring 360 which seats in a seating or groove 356 formed in shaft 354. Locking ring 360 abuts a radially inward projecting, annular flange 334 formed on an inner wall of shaft 330 (see FIG. 3C).

A forward end of shaft 330 can include an attachment or connector through which any of a number of accessories can be attached to connector 300. In the embodiment of FIGS. 3A through 3D, connector 300 includes a seating or groove 338 formed on an outward end thereof through which such accessories can be attached to connector 300. As clear to one skilled in the art, other attachment mechanisms as known in the art are suitable for use in attaching accessories to the connectors of the present invention. In the embodiment of FIGS. 3A through 3E, a cap member 370 is attached to connector 300 via a radially inward projecting, annular flange 372 which forms a snap fit with seating 338. Seating 338 (or other connection mechanism such as a flange similar to flange 352) can also be used in attaching accessories as discussed in connection with connector 350.

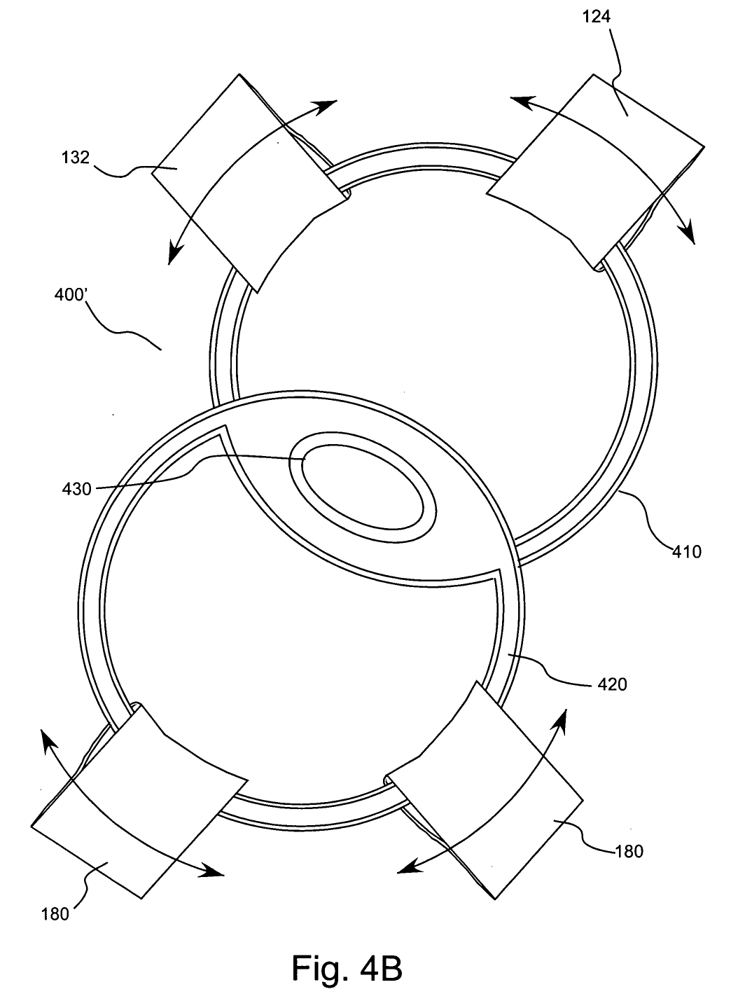

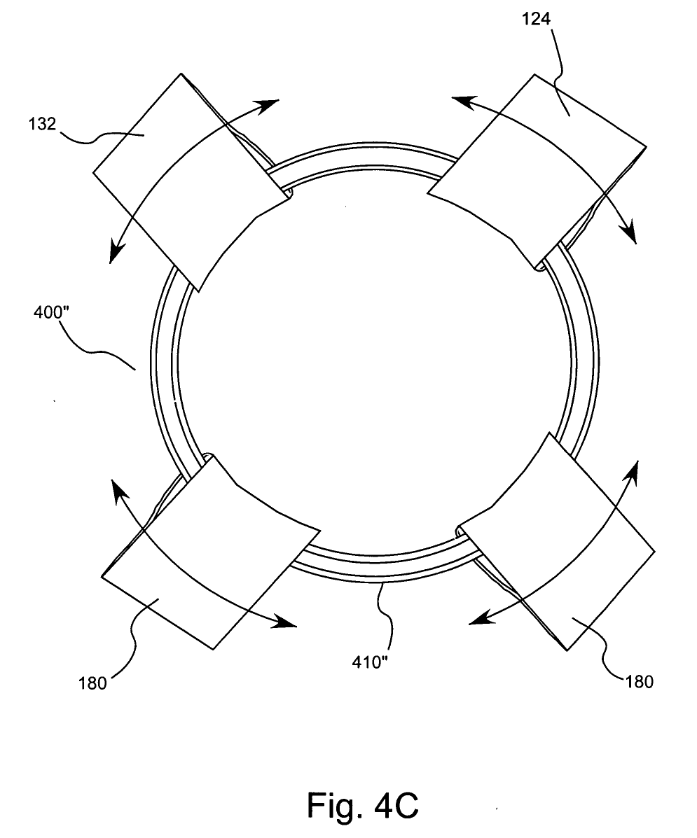

FIG. 4A illustrates another embodiment of a connector 400 of the present invention. Connector 400 includes a lower connecting member 320 which is generally identical to that of connector 300. However, rather than an upper connecting member as included in connector 300, connector 400 includes a preferably curved connecting member such as ring or loop member 410 to which rear strap 132 and front strap 124 are slidably attached. Ring member 410 need not be rotatably attached to lower connecting member 320. When a user wearing a safety harness of the present invention including connector 400 bends forward as illustrated in FIGS. 2C and 2D the strap ends of rear strap 132 and front strap 124 move (that is, slide) around the circumference of ring member 410, thereby preventing any substantial increase in tension in the straps of lower seat portion 112. FIG. 4B illustrates another embodiment of a connector 400′ in which two ring members 410 and 420 are connected (in a non-rotating connection by a connect 430. As described above, rear strap 132 and front strap 124 are slidably attached to ring member 410. In this embodiment, leg strap 180 is slidably attached at its first and second ends to ring member 420. FIG. 4C illustrates another embodiment of a connector 400″ of the present invention including a single ring member 410″ to which rear strap 132, front strap 124 and the ends of leg strap 180 are slidably attached.

As described above, tension in the straps of a safety harness of the present invention upon forward and rearward bending of a user can thus be reduced or generally eliminated in, for example, two general manners or combinations thereof. In that regard, one can connect in a generally fixed manner (that is, a non-sliding manner) strap ends of the upper torso portion to two upper connecting members. One can also connect in a generally fixed manner (that is, non-sliding manner) strap ends of the lower torso portion to two lower connecting members. The upper connecting member and the lower connecting member are then connected so that the upper connecting member is rotatable relative to the lower connecting member. Alternatively, one or more of the strap ends can be slidably attached to a connecting member. In one such embodiment, the connecting member includes an element (which can, for example, be a curved element) about which the a strap end is looped so that the strap end can slide along the element. In this embodiment, the connecting member need not be rotatable.

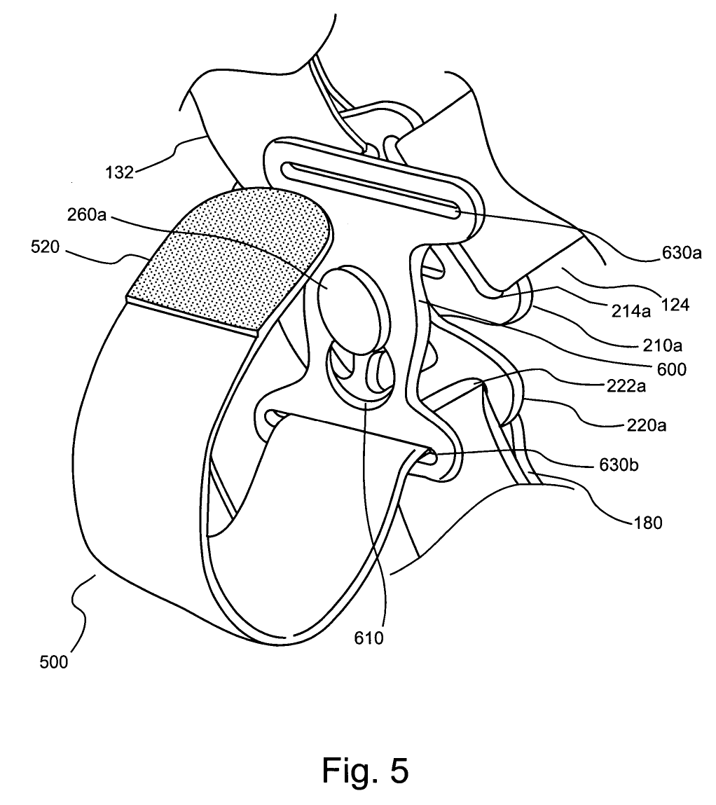

As also described above, various accessories can be attached to the connectors of the present invention. FIG. 5 illustrates the attachment of a strap 500, which can, for example, be formed into a loop and used to attached a tool belt, to connector 200a. In that regard, connector 200a includes a flange 260a attached to the outward end of shaft 230. A high-strength clip 600 (for example, fabricated from a polymeric material) snaps onto flange 260a via a slot 610 formed in clip 600. Strap 500 can, for example, be attached to clip 600 via slots 630a and 630b formed in clip 600. In the embodiment of FIG. 5, strap 500 is generally permanently attached to slot 630b (for example, via a sewn loop). Strap 500 can be passed through slot 630a to form a loop. The free end of strap 600 can, for example, include a hook-and-loop type fastening system 520 such as VELCRO®.

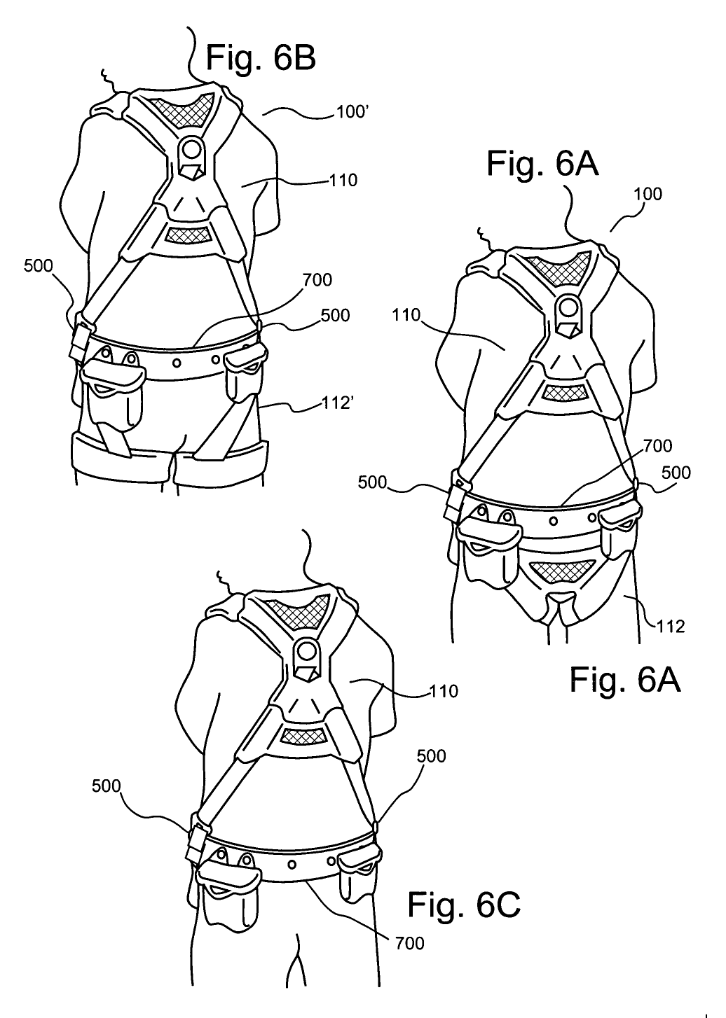

FIGS. 6A and 6B illustrate how connectors of the present invention of the present invention including upper and lower connecting members (such as upper connecting members 210a and 210b and lower connecting member 220a and 220b) can be used to readily fabricate modular full body safety harnesses in which various upper torso portions or modules can be mixed and matched with various lower seat portions or modules to easily and inexpensively fabricate a variety of harness for different uses and for different users. For example, using 10 different upper torso modules and 10 different lower seat modules, 100 different full body safety harnesses can be assembled. FIG. 6A illustrates a rear view of a user wearing full body safety harness 100 including upper torso module 110 and lower seat module 112 as described above. In FIG. 6A, the user is also wearing a tool belt 700 that is attached to safety harness 100 via looped straps 500 as described above. In FIG. 6B, the user is wearing a full body safety harness 100′ including upper torso module 110 and a different lower seat module 112′. Lower seat module 112′ includes leg straps 180′ and 190′ than encircle the legs of the user as is common in European safety harnesses. As illustrated in FIG. 6C, upper torso module 110 can be used without a lower seat module to, for example, act as suspenders to support tool belt 700. The modularity provided by the connectors of the present invention can substantially reduce the manufacturing costs in manufacturing a wide variety of harness types as compared to current manufacturing methods.

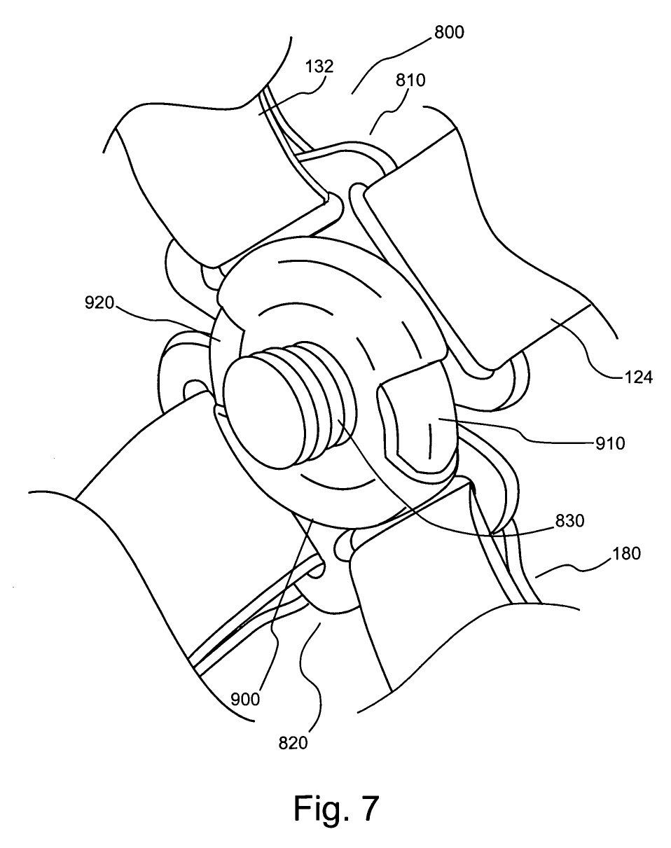

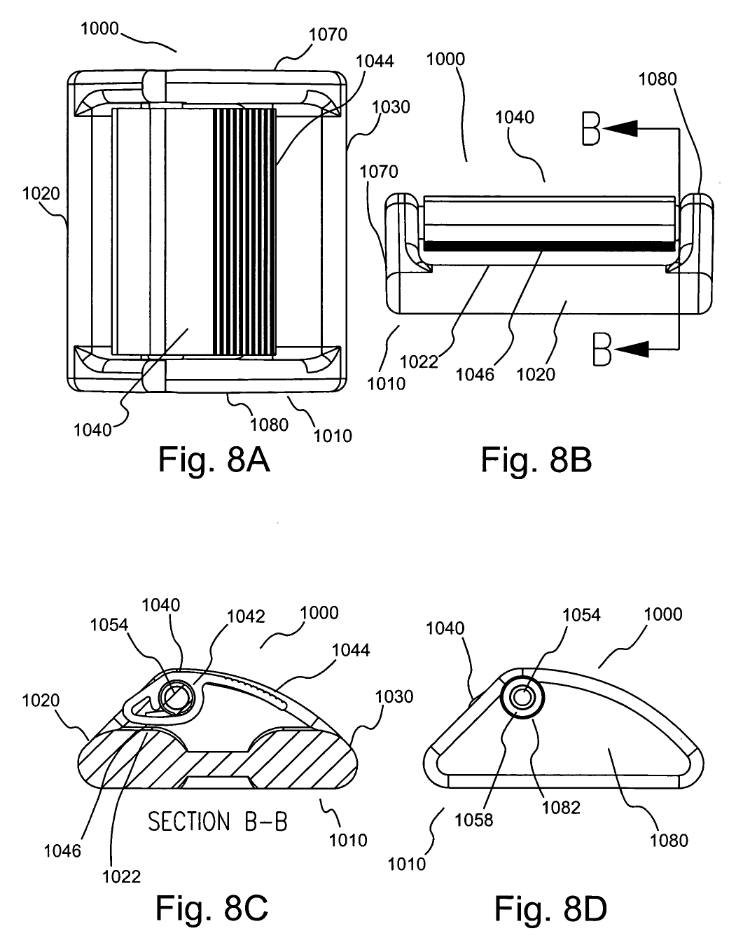

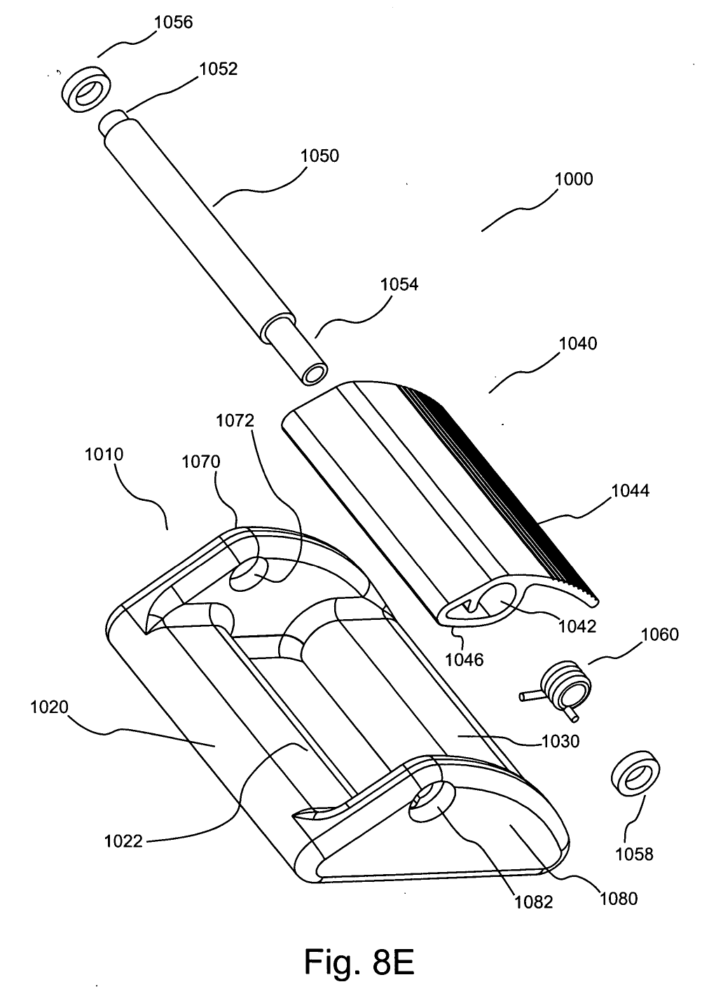

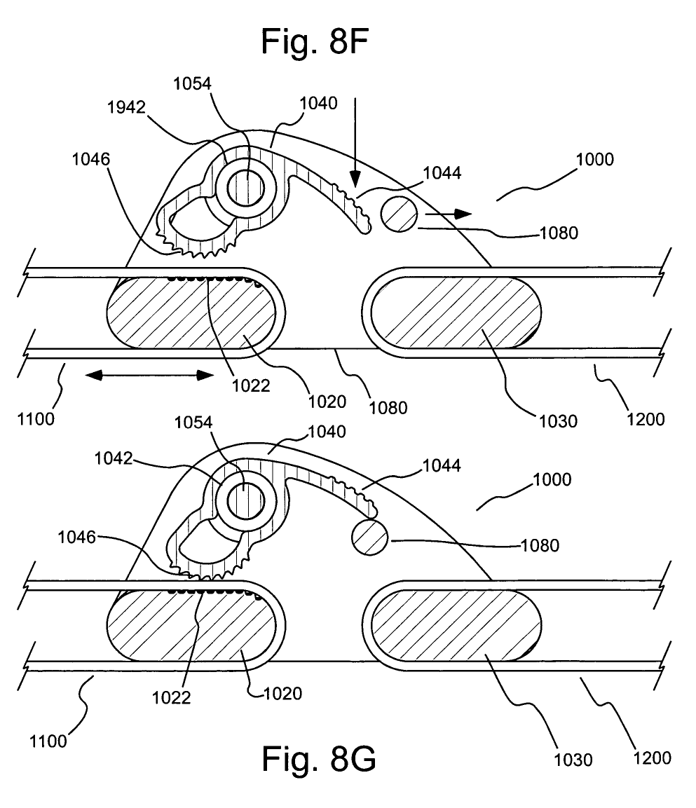

The connector for connecting upper torso modules and lower seat modules of the present invention can, for example, be fabricated to be disconnectable only by the manufacturer or can be made to be more readily disconnectable so that disconnection can, for example, be made by those in the field (for example, by an authorized safety officer). In that regard, FIG. 7 illustrates an embodiment of a connector 800 of the present invention that operates in a number of manners similarly to connector 200a. In that regard, connector 800 includes upper connecting member 810 and lower connecting member 820 that are rotatably connected via a shaft 830. Connector 800 also includes a release 900 operable to release upper connecting member 810 and lower connecting member 820 from connection with shaft 830 and thereby from connection with each other. Release 900 can include any of various release mechanisms known to those skilled in the art. In the embodiment of FIG. 8, release 900 includes release buttons 910 and 920 which are moved radially inward to cause disconnection. In one embodiment, each of buttons 910 and 920 must be actuated to cause disconnection to decrease the likelihood of accidental disconnection. A locking mechanism (for example, requiring an actuating key or other instrument) as known in the art can also be provided to ensure that disconnection is effected only by authorized personnel and that accidental disconnection does not occur.

In several embodiments, the harnesses of the present invention include one or more cam buckles that function as adjustment members such as adjustment members 126 and 136 to adjust the fit of the harnesses. The inventors of the present invention have discovered that such cam buckles provide a significant improvement in the ease of adjustment of the fit of one or more of the straps of the present invention as compared to adjustment mechanisms such as friction buckles previously used in safety harnesses. Moreover, the present inventors have discovered that cam buckles provide suitable locking strength such that the cam buckles do not open under loads experienced under normal conditions of use or under fall conditions.

FIGS. 8A through 8G illustrate one embodiment of a cam buckle 1000 suitable for use in harnesses of the present invention and in other safety harnesses. Buckle 1000 includes a base 1010 including a first strap support 1020 and a second strap support 1030. FIGS. 8F and 8G illustrate strapping sections 1100 and 1200 looped around first support 1010 and second support 1020, respectively. If desired or required by law, base 1010 can be fabricated from high-strength materials (which, can, for example, include various metals, high-strength polymeric materials, high-strength composite materials or combinations thereof) such that base 1010 provides a relatively high ultimate tensile load (for example, of at least 4000 pounds or at least 5000 pounds).

Buckle 1000 further includes a cam-shaped locking member 1040 that operates to lock strapping section 1100 in a desired position and to enable release of strapping section 1100 to allow adjustment of the fit of a harness by moving or sliding strapping section 1100 around or over support 1020. Locking member 1040 includes a passage 1042 through which a rod 1050 (around which locking member 1040 is rotatable) passes. Rod 1050 includes a first end section 1052 of generally reduced radius that passes through a passage or seating 1072 in a first side member 1070 of base 1010 and is held in position therein by bearing 1056. On the opposite end, rod 1050 includes a second end section 1054 of generally reduced radius that passes through a passage or seating 1082 in a second side member 1080 of base 1010 and is held in position by bearing 1058. A biasing element can be used to bias locking member in a locking position in which strap 1100 is prevented from moving or sliding relative to first strap support member 1020. In the embodiment of FIGS. 8A through 8G, the biasing element is a spring 1060 which is positioned within passage 1042. Second end section 1054 of rod 1050 passes through spring 1060 which closes tightly around second end section 1054 to bias locking member 1010 in a locking position as illustrated, for example, in FIG. 8G.

To place locking member 1040 in a release position as illustrated in FIG. 8F, a user applies a downward force (in the orientation of FIGS. 8F and 8G) to a lever arm 1044 of locking member 1040 to cause rotation of locking member 1040 around rod 1050 in a clockwise direction (once again in the orientation of FIGS. 8F and 8G). Rotation of locking member 1040 as illustrated in FIG. 8F, causes an abutment surface 1046 on an end of locking member 1040 generally opposite to lever arm 1044 to rotate out of contact with strapping section 1100, thereby releasing strap 1100 to enable adjustment thereof. Once strapping section 1100 is in adjusted to a desired position or fit, force is removed from lever arm 1044 so that locking member rotates around rod 1050 in a counterclockwise direction (in the orientation of FIGS. 8F and 8G) so that abutment surface 1046 rotate back into its biased position of contact with strapping section 1100. Strapping section 1100 is held or locked in position between abutment surface 1046 of locking member 1040 and an upper abutment surface 1022 of strap support 1020. As illustrated, for example, in FIGS. 8F and 8G, abutment surface 1046 and/or abutment surface 1022 can include knurling or ridges to assist in holding strapping section 1100 in a locked position.

A dual or double action can be required to place locking member 1040 in a release position. As illustrated in FIGS. 8F and 8G, for example, a moveable actuating, activating or abutment member 1080 can be placed in operative connection with lever arm 1044 to prevent movement of lever arm 1044 to a release position until abutment member 1080 is first moved to a release position (see FIG. 8F). Buckle 1000 can be fabricated in part or in whole from polymeric materials. In on embodiment base 1010 is fabricated from a polymeric material and locking member 1040 is fabricated from aluminum.

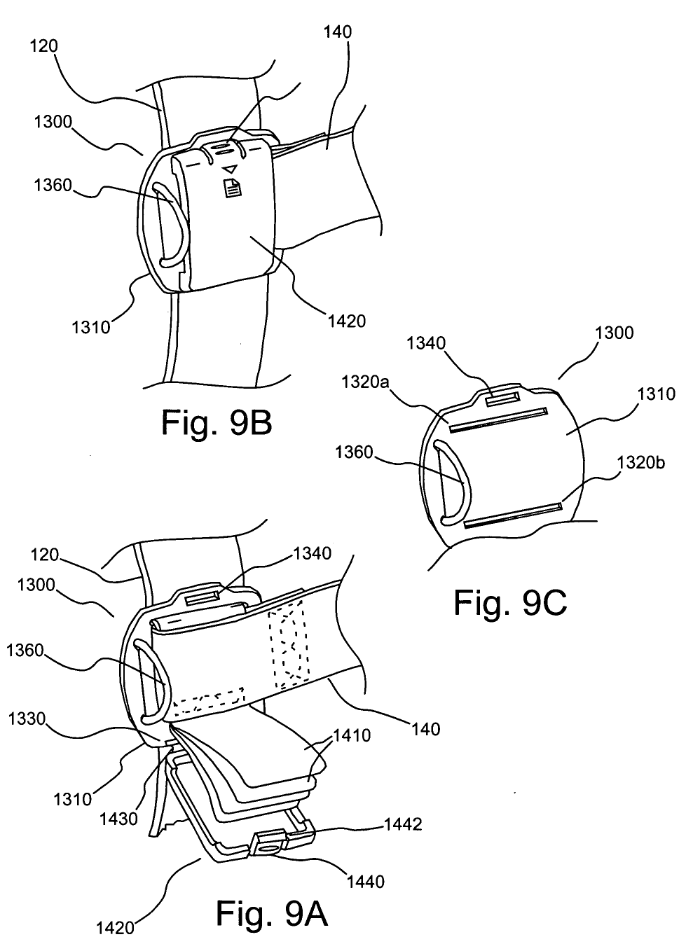

Under current law in the United States and other countries, a number of labels are required to be attached to safety harnesses. In currently available safety harnesses, label packs are typically sewn to the harness. Such label packs often become loose, damaged or lost. FIGS. 9A through 9C illustrate a connector 1300 of the present invention which can be used to attach a label pack 1400 and/or other items to safety harness 100 or to other safety harnesses. Connector 1300 can, for example, include a base 1310 which can, for example, be fabricated from a resilient polymeric material. Base 1310 includes an attachment mechanism for removable or non-removable attachment of base 1310 to harness 100. In the embodiment illustrated, for example, in FIGS. 9A through 9C, base 1310 includes two slots 1320a and 1320b through which harness strapping or webbing can passed to attach base 1310 to harness 100 or to another harness. As clear to one skilled in the art, many other types of attachments (either removable or non-removable) can be used. As illustrated, for example, in FIGS. 2A, 9A and 9B, shoulder strap 120 of harness 100 can be passed through slots 1320a and 1320b. Chest strap 140 can be looped around shoulder strap 120 in the area of shoulder strap 120 between slot 1320a and 1320, and chest strap 140 can then be sewn to itself as illustrated, for example, in FIGS. 9A and 9B.

Base 1310 further includes an attachment mechanism for attaching an item such as a label pack system 1400 and/or other items thereto. In the embodiment of FIGS. 9A through 9C, the item attachment mechanism includes a lower slot 1330 (see FIG. 9B) and an upper slot 1340. An item or a portion thereof to be attached to base 1310 via, for example, a snap fit with one or both of slots 1330 and 1340. As clear to one skilled in the art many other types of attachments can be used. In the embodiment of FIGS. 9A through 9C, label pack system 1400 includes various labels 1410 and a lid or closure 1420. Closure 1420 can, for example, be permanently or removably attached to base 1310 via lower slot 1330 via a snap fit as known in the art. Closure 1420 can alternatively be formed integrally with base 1310 or otherwise permanently or removably attached thereto. Closure 1420 can include a hinge 1430 (for example, a live hinge as known in the art) about which closure 1420 can rotate or hinge to an open position (as illustrated in FIG. 9A) and to a closed position (as illustrated in FIG. 9B). Closure 1420 can further include a releasable locking or closing mechanism 1440 which cooperates with upper slot 1340 to hold closure 1420 in a closed position. Application of force to closing mechanism 1440 by the user can release closing mechanism 1440. Closing mechanism, can for example, include a lever arm extending from closure 1420 with an abutment flange 1442 formed on an end thereof to abut an interior surface of upper slot 1340. Application of, for example, a downward force (in the orientation of FIG. 9B) can remove abutment flange 1442 from contact with base 1310 around the perimeter of slot 1340 and enable opening of closure 1420.

Labels 1410 can, for example, be permanently attached to base 1310 via stitching or via an adhesive. Labels 1410 can alternatively be attached to closure 1420. Base 1310 and closure 1420 act together to protect labels 1410 and prevent loosening, detachment or damage of labels 1410.

Connector 1300 can be used to attach other items such as a cell phone holder 1500 (see FIG. 2A) to harness 100 and to other harnesses. Cell phone holder 1500 can, for example, be attached to base 1310 using slots 1330 and 1340 or otherwise attached to base 1310. Connector 1300 can be used to attach many other devices including, but not limited to, a global positioning system devices, transponders, various communication devices, and/or sensors to, among other things, detect a fall.

In the embodiment of FIGS. 9A through 9C, connector 1300 further includes a lanyard attachment ring 1360, which can be used by a wearer of harness 100 to attach a lanyard (not shown) when the lanyard is not in use for fall protection. In that regard, such a lanyard is attached to D-ring 150 at a first end and to an anchor point at a second end when the lanyard is in use for fall protection. However, attachment ring 1360 can be used to attach the second end of the lanyard when the lanyard is not in use for fall protection (for example, when the wearer is walking around on the ground or on another surface) to keep the lanyard from dragging behind the wearer.

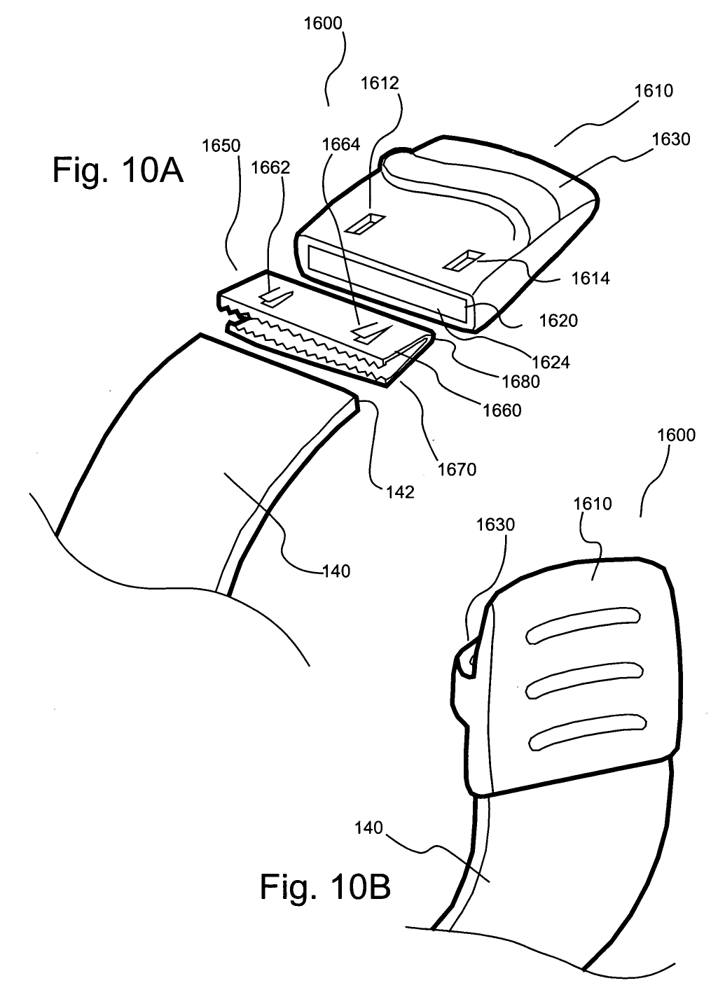

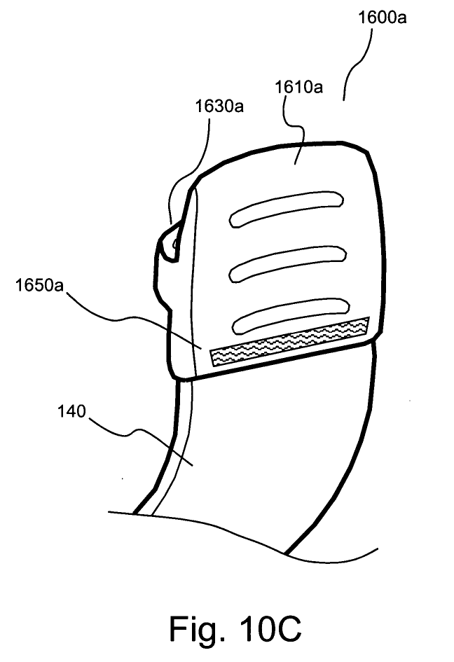

FIGS. 10A and 10B illustrate an embodiment of a harness strap end member 1600, which can operate, for example, to prevent fraying of a harness strap end such as an end 142 of chest strap 140. In one embodiment, end member 1600 includes a cap or housing 1610 and a clip 1650. Housing 1610 can, for example, be fabricated from a polymeric material. Clip 1650 is in the form of an alligator clip having a first or upper lever arm 1660, a second or lower lever arm 1670 and an intermediate hinging area 1680. Clip 1650 can, for example, be fabricated from an integral piece of a resilient polymeric material or an integral piece of a resilient metal (for example, spring steel). Each of lever arms 1660 and 1670 including gripping teeth on an end thereof opposite hinging area 1680.

Strap end 142 is first placed within clip 1650 so that, for example, strap end 142 abuts hinging area 1680. Clip 1650, with strap end 142 therein, is then pushed into an opening 1620 in housing 1610 which leads to a hollow portion or seating portion 1624 within housing 1610. Opening 1620 and seating 1624 are dimensioned to force lever arms 1660 and 1670 toward each other so that strap end 142 is securely gripped by the toothed ends of lever arms 1660 and 1670.

At least one of first lever arm 1660 and second lever arm 1670 can, for example, include a retaining mechanism to retain clip 1650 in operative connection with housing 1610. In the embodiment of FIGS. 10A and 10B, first lever arm 1660 includes abutment member 1662 and 1664 in the from of raised, resilient or flexible tabs. Tabs 1662 and 1664 are biased in an upward direction (in the orientation of FIG. 10A), but are forced downward as clip 1650 is pushed within seating 1624. Housing 1610 includes slots 1612 and 1614 into which tabs 1662 and 1664, respectively, can spring open. Once tabs 1662 and 1664 spring open or upward into slots 1612 and 1614, tabs 1662 and 1664 cooperate with slots 1612 and 1614 to prevent movement of clip 1650 and strap end 142 out of connection with housing 1610. Seating 1624 can include a rear abutment member or wall to facilitate positioning of tabs 1662 and 1664 in operative connection with slots 1612 and 1614. In the embodiment of FIGS. 10A and 10B, slots pass entirely through the wall of housing 1610, but such slots can pass only partially therethrough.

Housing 1610 can further include a retaining arm 1630 that can be slipped over or around a strap to hold end member 1600 in connection with the strap (see, for example, FIG. 2A). Retaining arm 1630 can, for example, be formed of a resilient material and create a gap between retaining arm 1630 and the surface of housing 1610 suitably dimensioned so that a gripping or biasing force is applied to the strap.

In the embodiment of FIGS. 10A and 10B, housing 1610 and clip 1650 are illustrated as separate, connectible elements. However, housing 1610 and clip 1650 can be integrated, for example comolded. Moreover, an end member or end housing such as housing 1650 can be attached to strap end 142 in manners other than using a clip such as clip 1650. For example, such a housing can be sewn to (via a stitching section 1650a as illustrated in FIG. 10C), riveted to and/or adhered to strap end 142. Such an end housing can also be attached via ultrasonic welding. Likewise, an end housing can be overmolded upon a strap end such as strap end 142 using overmolding techniques known in the art.

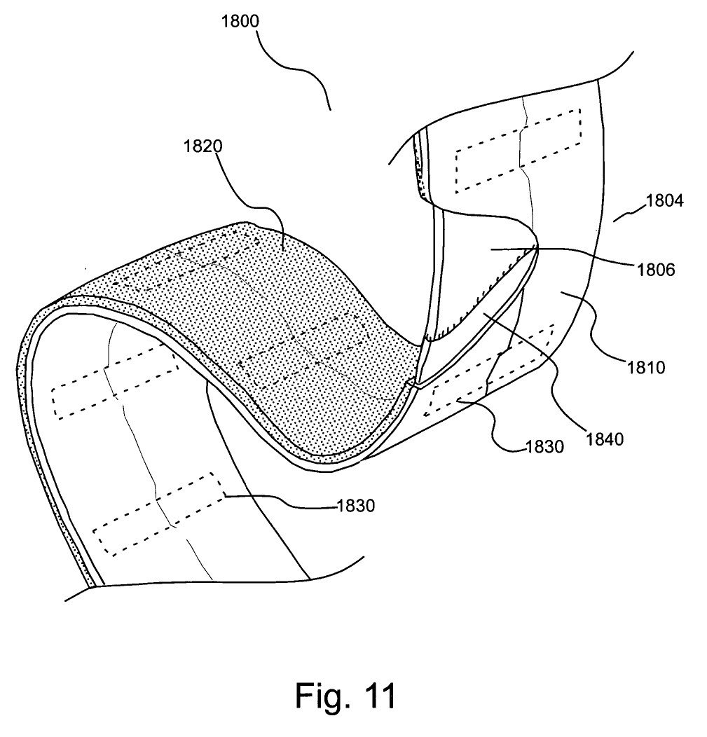

FIG. 11 illustrates an embodiment of harness webbing or strapping for use in harness 100 and other safety harnesses. Strap 1800 can, for example, include a generally tubular, outer shell 1804 fabricated from a flexible, high-strength material and a flexible inner member 1806 that can be a foam material as described generally in U.S. Pat. No. 6,739,427, assigned to the assignee of the present invention, the disclosure of which is incorporated herein by reference. In the embodiment of FIG. 11, outer shell 1804 includes a first or interior surface 1810 (that is, the surface that lays adjacent the wearer's body when the harness is worn) which is perceptibly different (for example, visibly different and/or different to the touch) from a second or outer surface 1820 thereof. Interior surface 1810 and outer surface 1820 can, for example, be of a different color, pattern and/or of a different texture. Inner surface 1810 can, for example, be woven to be of a different color from and softer than outer surface 1820 to increase the comfort of the user. Outer surface 1820 can be woven to be harder and/or more durable than inner surface 1810 to increase the wear life of the harness even in hostile environments such as experienced in welding operations. Additional or alternatively, materials, dyes or other elements can be added to at least one side of a manufactured harness strap to provide perceptibly different inner and outer surfaces of the harness strap.

Providing a harness including strapping sections having interior surfaces and exterior surfaces which are perceptibly different from each other facilitates donning the harness. In that regard, in currently available harnesses, it is quite difficult for a user to determine the interior and exterior surfaces of the harness, thereby making donning of the harness quite difficult. The interior surface and the exterior surface of the entire length of one or more straps (for example, the entire length of shoulder strap 120, including back strap 122 and front strap 124, and shoulder strap 130, including back strap 132 and front strap 134) can be perceptibly different as described above. Preferably, the interior surface and the exterior surface of a strap over a substantial portion (for example, at least 50%) of the length of the strap is perceptibly different such that donning is facilitated. In one embodiment, the entire length of each shoulder strap 120 and 130 and each leg strap 180 and 190 of safety harness 100 was formed of strapping having interior surfaces and exterior surfaces which were perceptibly different.

In the embodiment, of FIG. 11, inner member or material 1806 can, for example, be surrounded by a material 1840 having a highly visibly color that is different from each of inner surface 1810 and outer surface 1820 to act as a wear indicator. In that regard, should one of inner surface 1810 or outer surface 1820 wear through or tear, wear indicator material 1840 becomes visible and the harness can be taken out of service.

Inner surface 1810 and outer surface 1820 can, for example, be formed/woven separately and sewn together on the lateral sides of outer shell 1804 of strap 1800. Alternatively, inner surface 1810 and outer surface 1820 can be woven integrally using methods known in the weaving arts. In the embodiment of FIG. 11, strap 1800 includes spaced stitching sections 1830 that create a quilted effect and that can operate to prevent bunching or movement of inner member 1806 within outer sheath 1804. Spaced stitching sections 1830 can also create pockets for air flow.

The foregoing description and accompanying drawings set forth preferred embodiments of the invention at the present time. Various modifications, additions and alternative designs will, of course, become apparent to those skilled in the art in light of the foregoing teachings without departing from the scope of the invention. The scope of the invention is indicated by the following claims rather than by the foregoing description. All changes and variations that fall within the meaning and range of equivalency of the claims are to be embraced within their scope.

What is claimed is:

1. A full body safety harness to be worn by a person, comprising: an upper torso portion and a lower seat portion, the upper torso portion being operatively connected to the lower seat portion by a first connector on a first lateral side and a second connector on a second lateral side thereof, the first connector and the second connector enabling forward and rearward rotation of the upper torso portion relative to the lower seat portion without causing a significant increase in tension in the lower seat portion.

2. The full body safety harness of claim 1 wherein at least one of the first connector and the second connector comprises a rotating joint.

3. The full body safety harness of claim 1 wherein at least one of the first connector and the second connector comprises a ring member about which at least one strap of the upper torso portion or the lower seat portion is slidably attached.

4. The full body safety harness of claim 1 wherein the first connector comprises an upper ring member and a lower ring member, at least one end of a shoulder strap of the upper torso portion being slidably attached to the upper ring member, and at least one strap of leg strap of the lower seat portion being slidably attached to the lower ring member.

5. The full body safety harness of claim 1 wherein the upper torso portion comprises a first shoulder strap adapted to pass over a first shoulder of the person, a first end of the first shoulder strap adapted to extend over the front of the person to connect to the first connector, a second end of the first shoulder strap being adapted to extend over the back of the person to connect to the second connector, the upper torso portion further comprising a second shoulder strap adapted to pass over a second shoulder of the person, a first end of the second shoulder strap adapted to extend over the front of the person to connect to the second connector, a second end of the second shoulder strap being adapted to extend over the back of the person to connect to the first connector, the safety harness further comprising a first leg strap connected to the first connector and a second leg strap connected to the second connector, at least one of the first shoulder strap and the second shoulder strap including an adjustment mechanism to adjust the length thereof and at least one of the first leg strap and the second leg strap including an adjustment mechanism to adjust the length thereof.

6. The full body safety harness of claim 6 wherein the first shoulder strap comprises a first shoulder strap adjustment mechanism to adjust the length of the first shoulder strap, the second shoulder strap comprises a second adjustment mechanism to adjust the length of the second shoulder strap, the first leg strap comprises a first leg strap adjustment mechanism to adjust the length of the first leg strap and the second leg strap comprises a second leg strap adjustment mechanism to adjust the length of the second leg strap.

7. A full body safety harness to be worn by a person, comprising: an upper torso portion and a lower seat portion, the upper torso portion being operatively connected to the lower seat by a first rotating connector on a first lateral side and a second rotating connector on a second side thereof, the first rotating connector and the second rotating connector enabling rotation of the upper torso portion relative to the lower seat portion.

8. The full body safety harness of claim 7 wherein the first rotating connector is positioned to rotate about a point in the vicinity of the axis of rotation of the hips of the person when worn by the person and the second rotating connector is positioned to rotate about a point in the vicinity of the axis of rotation of the hips of the person when worn by the person.

9. The full body safety harness of claim 7 wherein the first rotating connector comprises an upper connective member and a lower connective member, the upper connective member being rotatably connected to the lower connective member.

10. The full body safety harness of claim 9 wherein the upper connective member is connected to the lower connective member by a shaft about which the upper connective member can rotate relative to the lower connective member.

11. The full body safety harness of claim 7 wherein the upper connective member comprises at least one attachment adapted to connect to harness strapping and the lower connective member comprises at least one attachment adapted to connect to harness strapping.

12. The full body safety harness of claim 7 wherein the upper connective member comprises at least two slots formed therein for attachment of harness strapping and the lower connective member comprises at least two slots formed therein of attachment of harness strapping.

13. The full body safety harness of claim 7 wherein the rotating connector further comprises an accessory connector attached to an outer surface thereof which is adapted to connect accessories to the full body safety harness.

14. The full body safety harness of claim 5 wherein the rotating connector further comprises a belt connector attached to an inner surface thereof which is adapted to connect the full body safety harness to a belt.

15. The full body safety harness of claim 14 wherein the belt is a safety positioning belt comprising an anchor attachment.

16. The full body safety harness of claim 14 wherein the belt is a tool belt.

17. The full body safety harness of claim 7 wherein the upper torso portion comprises a first shoulder strap adapted to pass over a first shoulder of the person, a first end of the first shoulder strap adapted to extend over the front of the person to connect to the first rotating connector, a second end of the first shoulder strap being adapted to extend over the back of the person to connect to the second rotating connector, the upper torso portion further comprising a second shoulder strap adapted to pass over a second shoulder of the person, a first end of the second shoulder strap adapted to extend over the front of the person to connect to the second rotating connector, a second end of the second shoulder strap being adapted to extend over the back of the person to connect to the first rotating connector.

18. The full body safety harness of claim 17 wherein the first shoulder strap and the second shoulder strap cross over each other in the back of the full body safety harness.

19. The full body safety harness of claim 17 wherein the lower torso portion comprises a first leg strap connected to the first rotating connector and a second leg strap connected to the second rotating connector.

20. The full body safety harness of claim 7 wherein the upper torso portion comprises a first shoulder strap adapted to pass over a first shoulder of the person, a first end of the first shoulder strap adapted to extend over the front of the person to connect to the first rotating connector, a second end of the first shoulder strap being adapted to extend over the back of the person to connect to the second rotating connector, the upper torso portion further comprising a second shoulder strap adapted to pass over a second shoulder of the person, a first end of the second shoulder strap adapted to extend over the front of the person to connect to the second rotating connector, a second end of the second shoulder strap being adapted to extend over the back of the person to connect to the first rotating connector, the safety harness further comprising a first leg strap connected to the first rotating connector and a second leg strap connected to the second rotating connector, at least one of the first shoulder strap and the second shoulder strap including an adjustment mechanism to adjust the length thereof and at least one of the first leg strap and the second leg strap including an adjustment mechanism to adjust the length thereof.

21. The full body safety harness of claim 20 wherein the first shoulder strap includes a first shoulder strap adjustment mechanism to adjust the length of the first shoulder strap, the second shoulder strap includes a second adjustment mechanism to adjust the length of the second shoulder strap, the first leg strap include a first leg strap adjustment mechanism to adjust the length of the first leg strap and the second leg strap includes a second leg strap adjustment mechanism to adjust the length of the second leg strap.

22. The full body safety harness of claim 1 further comprising a cam buckle in operative connection with at least one of strap section of the upper torso portion or the lower torso portion to adjust a fit of the strap section.

23. The safety harness of claim 22 wherein the cam buckle comprises a base and a locking member in moveable, operative connection with the base, the locking member including an abutment surface moveable into and out of contact with the strap.

24. The safety harness of claim 23 wherein the locking member is rotatably attached to the base and the abutment surface is biased in connection with the strap.

25. The safety harness of claim 24 wherein the base includes a strap support over which the strap passes, the abutment surface of the locking member being biased in connection with a first surface of the strap over a section of the strap wherein a second, opposing surface of the strap contacts the support member.

26. The safety harness of claim 25 wherein the locking member comprises a lever arm to which force is applied to move the locking member into a release position in which the abutment surface is out of contact with the strap.

27. The safety harness of claim 26 wherein the cam buckle comprises an activating mechanism that must be activated to enable the locking member to be moved to a release position.

28. The safety harness of claim 1 further comprising a connector attached to at least one strap section of the upper torso portion or the lower torso portion, the connector including a first attachment mechanism to attach the connector to the strap and a second attachment mechanism to attach an item to the connector.

29. The safety harness of claim 28 wherein the connector further comprises a base, the first attachment mechanism including a pair of slots formed in the base.

30. The safety harness of claim 29 wherein the connector further comprises a closure in operative connection with the base, the base and the enclosure at least partially enclosing at least one label when the closure in a closed state.

31. The safety harness of claim 30 wherein the closure includes a hinge about which the closure is rotatable to the closed state and to an open state, the at least one label being accessible when the closure is in an open state.

32. The safety harness of claim 31 wherein the closure includes a releasable locking mechanism to releasably cooperate with the base to hold the closure in a closed state.

33. The safety harness of claim 28 wherein the connector further comprises a closure in operative connection with a base, the base and the enclosure at least partially enclosing at least one label when the closure in a closed state.

34. The safety harness of claim 1 further comprising at least one strap section having an interior surface that is adjacent a wearer when the safety harness is worn and an exterior surface generally opposite the interior surface, the interior surface being perceptibly different from the exterior surface so that a wearer can distinguish the interior surface from the exterior surface.

35. The safety harness of claim 34 wherein the interior surface has a different color from the exterior surface.

36. The safety harness of claim 34 wherein the interior surface has a different texture from the exterior surface.

37. The safety harness of claim 34 wherein the interior surface is softer than the exterior surface.

38. The safety harness of claim 34 wherein the interior surface and the exterior surface are formed about generally tubular outer shell, the strap section further comprising a flexible inner material.

39. The safety harness of claim 37 wherein the exterior surface is more durable than the interior surface.

40. The safety harness of claim 1 further comprising at least one strap section have a first end; the first end including an end member in operative connection therewith, the end member comprising a retaining member to connect the end member to the at least one strap or to another strap of the harness.

41. The safety harness of claim 40 wherein the retaining member includes an extending arm to extend around the another strap.

42. The safety harness of claim 41 wherein the extending member is biased against the another strap when extending around the another strap.

43. A full body safety harness to be worn by a person, comprising: an upper torso portion and a lower seat portion, the upper torso portion being operatively connected to the lower seat by at least one connector adapted to enable rotation of the upper torso portion over a range of positions forward and rearward relative to the lower seat portion without causing significant tension in the lower seat portion.

44. A full body safety harness to be worn by a person, comprising: an upper torso portion and a lower seat portion, the upper torso portion being removably connected to the lower seat portion via a first connector on a first lateral side of the harness and a second connector on a second lateral side of the harness.

45. The safety harness of claim 44 wherein the first connector comprises an first upper connecting member to which at least one strap of the upper torso portion is connected and a first lower connecting member to which at least one strap of the lower torso portion is connected, the first upper connecting member and the first lower connecting member being adapted to be placed in operative connection, and the second connector comprises a second upper connecting member to which at least one strap of the upper torso portion is connected and a second lower connecting member to which at least one strap of the lower torso portion is connected, the second upper connecting member and the second lower connecting member being adapted to be placed in operative connection.

46. The safety harness of claim 45 wherein the first upper connecting member and the first lower connecting member are adapted to be placed in operative connection such that the first upper connecting member is rotatable about an axis relative to the first lower connecting member, and the second upper connecting member and the second lower connecting member are adapted to be placed in operative connection such that the second upper connecting member is rotatable about an axis relative to the second lower connecting member.

47. A method of fabricating a full body safety harness, comprising:
forming an upper torso portion of the safety harness;
forming separately from the upper torso portion a lower seat portion; and
connecting the upper torso portion to the lower seat portion via a first connector on a first lateral side of the full body safety harness and a second connector on a second side of the safety harness, wherein the first connector comprises an first upper connecting member to which at least one strap of the upper torso portion is connected and a first lower connecting member to which at least one strap of the lower seat portion is connected, the first upper connecting member and the first lower connecting member being adapted to be placed in operative connection, and the second connector comprises a second upper connecting member to which at least one strap of the upper torso portion is connected and a second lower connecting member to which at least one strap of the lower seat portion is connected, the second upper connecting member and the second lower connecting member being adapted to be placed in operative connection.

48. The method of claim 24 further comprising:
forming a plurality of upper torso portions, each have a unique configuration; and
selecting one of the plurality of upper torso portions to be connected to the lower seat portion.

49. The method of claim 24 further comprising:
forming a plurality of lower seat portions, each have a unique configuration; and
selecting one of the plurality of lower seat portions to be connected to the upper torso portion.

50. A safety harness to be worn by a person, comprising at least one strap section, the strap section including a cam buckle in operative connection therewith to adjust a fit of the strap section.

51. The safety harness of claim 50 wherein the can buckle comprises a base and a locking member in moveable, operative connection with the base, the locking member including an abutment surface moveable into and out of contact with the strap.

52. The safety harness of claim 52 wherein the locking member is rotatably attached to the base and the abutment surface is biased in connection with the strap.

53. The safety harness of claim 52 wherein the base includes a strap support over which the strap passes, the abutment surface of the locking member being biased in connection with a first surface of the strap over a section of the strap wherein a second, opposing surface of the strap contacts the support member.

54. The safety harness of claim 53 wherein the locking member comprises a lever arm to which force is applied to move the locking member into a release position in which the abutment surface is out of contact with the strap.

55. The safety harness of claim 54 wherein the cam buckle comprises an activating mechanism that must be activated to enable the locking member to be moved to a release position.

56. A connector for use in connection with a strap of a safety harness, the connector including a first attachment mechanism to attach the connector to the strap and a second attachment mechanism to attach an item to the connector.

57. The connector of claim 56 further comprising a base, the first attachment mechanism including a pair of slots formed in the base.

58. The connector of claim 57 further comprising a closure in operative connection with the base, the base and the enclosure at least partially enclosing at least one label when the closure in a closed state.

59. A label pack system for use in connection with a safety harness including a strap, the label pack system comprising: a base including an attachment mechanism to attach the base to the strap of the safety harness and a closure in operative connection with the base, the base and the enclosure at least partially enclosing at least one label when the closure in a closed state.

60. The label pack system of claim 59 wherein the closure includes a hinge about which the closure is rotatable to the closed state and to an open state, the at least one label being accessible when the closure is in an open state.

61. The label pack system of claim 60 wherein the closure includes a releasable locking mechanism to releasably cooperate with the base to hold the closure in a closed state.

62. A safety harness comprising a strap section having an interior surface that is adjacent a wearer when the safety harness is worn and an exterior surface generally opposite the interior surface, the interior surface being perceptibly different from the exterior surface so that a wearer can distinguish the interior surface from the exterior surface.

63. The safety harness of claim 62 wherein the interior surface has a different color from the exterior surface.

64. The safety harness of claim 62 wherein the interior surface has a different texture from the exterior surface.

65. The safety harness of claim 62 wherein the interior surface is softer than the exterior surface.

66. The safety harness of claim 62 wherein the interior surface and the exterior surface are formed about generally tubular outer shell, the strap section further comprising a flexible inner material.

67. The safety harness of claim 65 wherein the exterior surface is more durable than the interior surface.

68. A safety harness comprising at least one strap section have a first end; the first end including an end member in operative connection therewith, the end member comprising a retaining member to connect the end member to the strap or to another strap of the harness.

69. The safety harness of claim 68 wherein the retaining member includes an extending arm adapted to extend around the strap or around the another strap.

70. The safety harness of claim 69 wherein the extending member is adapted to be biased against the strap or against the another strap when extending around the strap or around the another strap.
